(19) United States
(12) Patent Application Publication
(10) Pub. No.: US 2023/0343468 A1
(43) Pub. Date: Oct. 26, 2023

(54) AUTOMATED INDIVIDUALIZED RECOMMENDATIONS FOR MEDICAL TREATMENT

(71) Applicant: xCures, Inc., Oakland, CA (US)

(72) Inventor: Mark A. Shapiro, Durham, NC (US)

(21) Appl. No.: 18/127,866

(22) Filed: Mar. 29, 2023

Related U.S. Application Data
(63) Continuation of application No. PCT/US2021/052400, filed on Sep. 28, 2021.
(60) Provisional application No. 63/084,984, filed on Sep. 29, 2020.

(51) Int. Cl. G16H 50/70; G06F 40/205; G06F 40/30; G06F 40/166; G06F 40/284

(52) U.S. Cl. CPC G16H 50/70; G06F 40/205; G06F 40/30; G06F 40/166; G06F 40/284; G16H 70/60

(57) ABSTRACT

Provided herein are systems and methods for automated generation of individual recommendations for medical treatment. The system and methods may ingest information from a variety of sources (e.g., clinical trials, tumor boards, case studies, etc.) and, based on this information, and a case summary provided by the physician, generate a ranked list of potential treatment options that are matched to the particular situation of a patient.

### CROSS-REFERENCE

This application is a continuation of International Application No. PCT/US2021/052400, filed Sep. 28, 2021, which claims the benefit of U.S. Provisional Patent Application No. 63/084,984, filed Sep. 29, 2020, each of which is incorporated by reference herein in its entirety.

### BACKGROUND

The present disclosure provides methods and system for addressing challenges doctors may face when treating patients with complex disease etiologies, such as cancer. A subject (e.g., patient) with cancer can have multiple genomic abnormalities—generally somatic, but sometimes germline as well—that interact in complex ways with environmental factors to produce the disease state. All patients may present to their medical professionals with their own distinct sets of comorbidities, histories of prior treatments, etc., making every case unique.

For many diseases, especially chronic ones such as type 2 diabetes (T2D) or congestive heart failure (CHF), there may be a long period of disease treatment where physicians adhere to strict treatment guidelines that apply broadly to large, fairly homogeneous cohorts, with little intra-cohort variation with respect to the disease state. But even with such diseases, the end stage of treating such patients, which often leads to multiple organ failure at differing rates, may force the practitioner to adjust treatment on a per-patient basis. Hence, one finds clinical trials such as clinicaltrials.gov/ct2/show/NCT01807221, which address patients with heart failure, diabetes, and kidney failure simultaneously.

While cancer is discussed throughout herein, the methods and embodiments disclosed herein are illustrative only and may apply to related domains as well. Cancer may be a particularly illustrative domain because of the rapid progress from guidelines-based medicine to individualized medicine, requiring knowledge of disease state, comorbidities, genomics, and other terms and topics.

### SUMMARY

In many clinically delineated stages of disease, there may be well established clinical guidelines. For example, the National Comprehensive Cancer Network (NCCN) publishes detailed flowcharts for disease state for most major types of cancer every one to three years. But when the standard of care has been exhausted, physicians may be left with no guidance on treatment for their patients, and they may be required to do research on their own.

It may be very difficult for even expert practitioners to keep up to date with all the available literature on clinical trials, case reports, tumor board discussions, and other sources of potential treatment options.

The present disclosure provides methods and systems that act as an intelligent assistant that can digest all information from a variety of sources (clinical trials, tumor boards, case summaries, patient reported outcomes, etc.), analyze an individual patient's case summary, and rank order treatment options based on features of the patient's case and the specifics of the treatments' applicability.

With this tool, physicians can find the right treatments, allowing them to prescribe therapies off-label and/or prescribe treatments through expanded access, alone or in combination, without their patients needing to travel to a clinical trial site. This can be done directly by the physician, or by the physician and patient participating in a decentralized trial.

A physician can access these potential therapies via the system of the present invention. They may do so by entering data about the patient's case history into the system, including patient status, comorbidities, genomics and other biomarkers, past treatments, etc.

The system may have previously ingested information on myriad clinical trials, tumor boards, case studies, etc. Based on this information, plus the case summary provided by the physician, the methods and systems of the present disclosure may produce a ranked list of potential treatment options that are matched to the particular situation of the patient. These may be considered singly or in combination by the physician as good starting points for treatment. Treatments likely to be ineffective may be dropped from the list, and treatments likely to be most effective may be promoted to the top of the list.

The methods and systems provided herein may offer numerous advantages over existing methods and systems. For example, methods that use both imaging data and non-image-based data in a clinical decision support system (CDSS) can help guide treatment for a patient. In these methods, the guidelines generated for a specific patient may be created in part by matching against a library of prior patients with similar clinical characteristics. For example, Natural Language Processing (NLP) may be used to extract features of the case report of the current patient, and to compare those to features of prior patients to find those prior patients who are closest to the current patient by some metric in the feature space. However, a limitation of such methods may be that they work by parameterizing existing guidelines. They may fall short and may not be applicable for domains where guidelines do not exist, such as late-stage cancer. Furthermore, these methods may be limited to simple mapping of terms between systems; there is no capability to cluster terms into higher-level concepts.

Other methods may extract data via NLP for use with guidelines, such as for determining whether information contained in the relevant data elements complies with a guideline. But such methods may not pertain to customizing or altering the guidelines, nor to developing treatment plans for a patient.

Thus, it can be seen that, while automated approaches for using NLP and related technologies may be developed to support and validate guidelines usage in standard clinical practice, there may be no similar automated approaches for assisting physicians and other practitioners with treatment selection where treatment needs have progressed beyond where guidelines can support the physician (for example, in cancer treatment where the standard of care has been exhausted).

It may be very difficult for even expert practitioners to keep up to date with all the available literature on clinical trials, case reports, tumor board discussions, and other sources of potential treatment options and adapt that information to individual care for patients that do not conform to existing guidelines. Thus, having an intelligent assistant that can digest all of this information, analyze an individual patient's case summary, and rank order treatment options based on features of the patient's case and the specifics of the treatments' applicability can greatly aid physicians in their daily work. Thus, recognized herein is an urgent need for methods and systems of the present disclosure, which may address at least the abovementioned problems.

In an aspect, the present disclosure provides a computer-implemented method for generating an individual recommendation for medical treatment of a subject, the method comprising: (a) receiving, from a first set of distinct sources, first information relating to a set of diseases or disorders encompassing a medical domain; (b) processing the first information relating to the set of diseases or disorders to generate a first document corpus, wherein processing the first information comprises parsing structured information or textual information of the first information; (c) receiving, from a second set of distinct sources, second information relating to a disease or disorder of the subject, wherein the second information comprises a clinical information of the subject; (d) processing the second information relating to the disease or disorder of the subject to generate a second document corpus, wherein processing the second information comprises parsing structured information or textual information of the second information; and (e) generating a ranked set of candidate treatments for treating the disease or disorder of the subject, based at least in part on processing the first document corpus with the second document corpus.

In some embodiments, (a) comprises receiving, from a remote server, the first information relating to the set of diseases or disorders encompassing the medical domain. In some embodiments, (c) comprises receiving, from a remote server, the second information relating to the disease or disorder of the subject.

In some embodiments, the disease or disorder is cancer. In some embodiments, the cancer is selected from the group consisting of breast cancer, colorectal cancer, brain cancer, leukemia, lung cancer, skin cancer, liver cancer, pancreatic cancer, lymphoma, esophageal cancer, and cervical cancer.

In some embodiments, the first information relating to the set of diseases or disorders comprises clinical trial information, a tumor board discussion, a case summary or report, and/or outcomes reported by subjects. In some embodiments, the second information relating to the disease or disorder of the subject comprises diagnosis, stage and grade of disease, medications, vitals, laboratory results, clinical trial information, tumor board discussions, a case summary or report, and/or an outcome reported by the subject. In some embodiments, the clinical trial information is received from a clinical trial database. In some embodiments, the clinical trial database comprises a National Clinical Trial repository. In some embodiments, the clinical trial information comprises at least one of clinical trials for specific treatments for the disease or disorder, information about trial arms, information about control arms, and inclusion or exclusion criteria for clinical trials. In some embodiments, the tumor board discussion comprises information relating to at least one of tradeoffs, inclusion or exclusion criteria, and efficacy for a plurality of candidate treatments. In some embodiments, the tumor board discussion is a virtual tumor board discussion. In some embodiments, the clinical information of the subject comprises a case summary of the disease or disorder of the subject.

In some embodiments, the case summary is prepared by a health care provider of the subject. In some embodiments, the health care provider comprises a physician. In some embodiments, the physician comprises an oncologist. In some embodiments, the case summary comprises structured data, unstructured data, or a combination thereof. In some embodiments, the case summary is conveyed from an electronic health record system. In some embodiments, the case summary comprises at least one of genomic features of the subject, treatment options for the subject, and tumor load of the subject.

In some embodiments, (b) further comprises parsing the structured information or textual information of the first information according to an ontology of treatment questions. In some embodiments, the ontology comprises at least one of subject features, disease state, and types of treatments. In some embodiments, (d) further comprises parsing the structured information or textual information of the second information according to an ontology of treatment concepts. In some embodiments, the ontology comprises at least one of concepts of the subject, disease state, and types of treatments.

In some embodiments, (b) further comprises parsing the structured information or textual information of the first information to discover concepts pertaining to at least one topic selected from clinical trial information, a tumor board discussion, a case summary or report, and outcomes reported subjects. In some embodiments, (d) further comprises parsing the structured information or textual information of the second information to discover concepts pertaining to at least one topic selected from diagnosis, stage and grade of disease, medications, vitals, laboratory results, clinical trial information, a tumor board discussion, a case summary or report, and an outcome reported by the subject.

In some embodiments, (b) further comprises generating a topic space for documents received from the first set of distinct sources. In some embodiments, the topic space comprises a plurality of hierarchical topic spaces. In some embodiments, the topic space is associated with a disease state or a treatment for the disease state. In some embodiments, (d) further comprises generating a topic space for documents received from the second set of distinct sources. In some embodiments, the topic space comprises a plurality of hierarchical topic spaces. In some embodiments, the topic space is associated with a disease state or a treatment for the disease state.

In some embodiments, (b) further comprises associating a topic with a specific document received from a distinct source of the first set of distinct sources. In some embodiments, (d) further comprises associating a topic with a specific document received from a distinct source of the second set of distinct sources.

In some embodiments, (b) further comprises parsing the structured information or textual information of the first information using one or more algorithms selected from the group consisting of a text recognition algorithm, a regular expressions algorithm, a pattern recognition algorithm, an imaging recognition algorithm, a natural language processing algorithm, an optical character recognition algorithm, a term frequency-inverse document frequency (TF-IDF) algorithm, and a bag-of-words algorithm. In some embodiments, (d) further comprises parsing the structured information or textual information of the second information using one or more algorithms selected from the group consisting of a text recognition algorithm, a regular expressions algorithm, a pattern recognition algorithm, an imaging recognition algorithm, a natural language processing algorithm, an optical character recognition algorithm, a term frequency-inverse document frequency (TF-IDF) algorithm, and a bag-of-words algorithm.

In some embodiments, (b) further comprises determining, based at least in part on the parsing in (b), whether the structured information or textual information of the first information corresponds to a clinical trials database, a clinical trial arm description, a genomics database, a clinical care guideline document, a case series document, a drug database, an imaging report, a pathology report, a clinic note, a progress note, a genomics report, a laboratory test report, a diagnostic report, or a prognostic report. In some embodiments, (d) further comprises determining, based at least in part on the parsing in (d), whether the structured information or textual information of the second information corresponds to an imaging report, a pathology report, a clinic note, a progress note, a genomics report, a laboratory test report, a diagnostic report, or a prognostic report.

In some embodiments, parsing the structured information or textual information of the first information comprises at least one of case converting the structured information or textual information of the first information, removing special characters or stop words from the structured information or textual information of the first information, tokenizing the structured information or textual information of the first information, and parsing the structured information or textual information of the first information using a parser. In some embodiments, parsing the structured information or textual information of the second information comprises at least one of case converting the structured information or textual information of the second information, removing special characters or stop words from the structured information or textual information of the second information, tokenizing the structured information or textual information of the second information, and parsing the structured information or textual information of the second information using a parser.

In some embodiments, parsing the structured information or textual information of the first information comprises filtering the structured information or textual information of the first information for a disease state, a treatment for the disease state, or clinical trials associated with the disease state or the treatment for the disease state. In some embodiments, parsing the structured information or textual information of the second information comprises filtering the structured information or textual information of the second information for a disease state, a treatment for the disease state, or clinical trials associated with the disease state or the treatment for the disease state.

In some embodiments, parsing the structured information or textual information of the first information comprises extracting and standardizing inclusion or exclusion criteria. In some embodiments, parsing the structured information or textual information of the second information comprises extracting and standardizing inclusion or exclusion criteria.

In some embodiments, parsing the structured information or textual information of the first information comprises labeling the structured information or textual information of the first information with labels. In some embodiments, the labels comprise information pertaining to a disease, a treatment, an inclusion, or an exclusion. In some embodiments, parsing the structured information or textual information of the second information comprises labeling the structured information or textual information of the second information with labels. In some embodiments, the labels comprise information pertaining to a disease, a treatment, an inclusion, or an exclusion.

In some embodiments, parsing the structured information or textual information of the first information comprises performing named entity recognition. In some embodiments, performing the named entity recognition comprises at least one of ontology mapping, speech tagging, and entity type tagging. In some embodiments, parsing the structured information or textual information of the second information comprises performing named entity recognition. In some embodiments, performing the named entity recognition comprises at least one of ontology mapping, speech tagging, and entity type tagging.

In some embodiments, (b) further comprises generating a set of sub-corpuses from the first document corpus. In some embodiments, (d) further comprises generating a set of sub-corpuses from the second document corpus.

In some embodiments, (b) further comprises performing topic modeling. In some embodiments, the topic modeling in (b) comprises use of at least one of Biterm Topic Modeling (BTM), Latent Dirichlet Allocation (LDA), and Term Frequency-Inverse Document Frequency (TF-IDF) analysis. In some embodiments, the topic modeling in (b) comprises use of the LDA or TF-IDF analysis. In some embodiments, the topic modeling in (b) comprises using the topic modeling to generate ngrams of frequently occurring word combinations in the first information. In some embodiments, the frequently occurring word combinations comprise single words, word pairs, triplets, or a combination thereof. In some embodiments, the ngrams comprise a frequency of occurrence of the frequently occurring word combinations. In some embodiments, the topic modeling in (b) comprises partitioning the first document corpus into a set of topics or subtopics. In some embodiments, the partitioning comprise use of a hyperparameter. In some embodiments, the hyperparameter is received from a human user. In some embodiments, the topic modeling in (b) comprises associating relationships between ngrams and treatments, ngrams and disease state, ngrams and treatment rationales, or a combination thereof. In some embodiments, associating the relationships comprises applying a chain rule analysis to account for interaction terms. In some embodiments, the chain rule analysis comprises performing matrix multiplication.

In some embodiments, (e) further comprises mapping the ngrams of at least one of the first information and the second information to a set of candidate treatments, and generating the ranked set of candidate treatments based at least in part on the mapping. In some embodiments, the mapping comprises partitioning at least one of the first document corpus and the second document corpus based on a topic. In some embodiments, the mapping comprises computing a weight matrix, and generating the ranked set of candidate treatments based at least in part on the weight matrix. In some embodiments, the mapping comprises use of a similarity matrix to account for at least partial mismatches. In some embodiments, the mapping comprises performing matrix multiplication using the similarity matrix. In some embodiments, the similarity matrix comprises a treatment similarity matrix comprising component metrics indicative of pairwise overlap between candidate treatments in a clinical trial, evaluated over a space of a plurality of clinical trials. In some embodiments, the component metrics comprise a member selected from the group consisting of Jaccard similarity between candidate treatments, cosine similarity between candidate treatments, Jaro-Winkler (J-W) distance between candidate treatments, and Jaccard syllable similarity between candidate treatments. In some embodiments, the component metrics comprise at least two members selected from the group consisting of Jaccard similarity between candidate treatments, cosine similarity between candidate treatments, Jaro-Winkler (J-W) distance between candidate treatments, and Jaccard syllable similarity between candidate treatments. In some embodiments, the method further comprises calculating an ensemble score for at least two treatment similarity matrices. In some embodiments, calculating the ensemble score comprises performing a dimensionality analysis. In some embodiments, the dimensionality analysis is selected from the group consisting of principal component analysis (PCA), t-distributed stochastic neighbor embedding (t-SNE), and uniform manifold approximation and projection (UMAP), and human supervision. In some embodiments, the similarity matrix comprises a disease similarity matrix comprising component metrics indicative of pairwise overlap between diseases in a clinical trial, evaluated over a space of a plurality of clinical trials. In some embodiments, the component metrics comprise a member selected from the group consisting of Jaccard similarity between diseases, cosine similarity between diseases, Jaro-Winkler (J-W) distance between diseases, and Jaccard syllable similarity between diseases. In some embodiments, the component metrics comprise at least two members selected from the group consisting of Jaccard similarity between diseases, cosine similarity between diseases, Jaro-Winkler (J-W) distance between diseases, and Jaccard syllable similarity between diseases. In some embodiments, the method further comprises calculating an ensemble score for at least two disease similarity matrices. In some embodiments, calculating the ensemble score comprises performing a dimensionality analysis. In some embodiments, the dimensionality analysis is selected from the group consisting of principal component analysis (PCA), t-distributed stochastic neighbor embedding (t-SNE), and uniform manifold approximation and projection (UMAP), and human supervision. In some embodiments, the mapping comprises using latent semantic analysis. In some embodiments, the mapping comprises performing a plurality of mappings comprising at least a first mapping from the ngrams to a topic, subtopic, or disease, and a second mapping from the topic, the subtopic, or the disease to the set of candidate treatments.

In some embodiments, (e) further comprises combining outputs from a plurality of mappings, and generating the ranked set of candidate treatments based at least in part on the combined outputs. In some embodiments, combining the outputs comprises summing the outputs from the plurality of mappings. In some embodiments, combining the outputs comprises using a set of weights to calculate a weighted sum of the outputs from the plurality of mappings. In some embodiments, combining the outputs comprises normalizing or scaling the set of weights. In some embodiments, the set of weights comprises values between 0 and 1. In some embodiments, the set of weights is adjusted using a training set. In some embodiments, the set of weights is adjusted by XGBoost, Bayesian rejection sampling, Thompson Sampling, upper confidence bound sampling, or knowledge gradient sampling. In some embodiments, the set of weights is adjusted based on a distance metric between a model-predicted treatment ranking and an observed treatment ranking. In some embodiments, the distance metric comprises a Kendall tau distance.

In some embodiments, processing the first document corpus with the second document corpus in (e) comprises comparing the first document corpus and second document corpus to each other.

In some embodiments, the method further comprises performing at least one iteration of (a) and (b) to incorporate new or updated medical information into the first document corpus. In some embodiments, (b) comprises using a Bayesian update process to incorporate the new or updated medical information into the first document corpus. In some embodiments, (b) comprises, subsequent to the subject being followed to a specified endpoint, incorporating the new or updated medical information of the subject into the first document corpus, thereby allowing additional subjects to benefit therefrom. In some embodiments, the method further comprises performing (c) to (e) for an additional subject in need of an individual recommendation for medical treatment.

In another aspect, the present disclosure provides a system for generating an individual recommendation for medical treatment of a subject, comprising: a database that is configured to (i) receive from a first set of distinct sources, first information relating to a set of diseases or disorders encompassing a medical domain, and (ii) receive from a second set of distinct sources, second information relating to a disease or disorder of the subject, wherein the second information comprises a clinical information of the subject; and one or more computer processors operatively coupled to the database, wherein the one or more computer processors are individually or collectively programmed to: (a) process the first information relating to the set of diseases or disorders to generate a first document corpus, wherein processing the first information comprises parsing structured information or textual information of the first information; (b) process the second information relating to the disease or disorder of the subject to generate a second document corpus, wherein processing the second information comprises parsing structured information or textual information of the second information; and (c) generate a ranked set of candidate treatments for treating the disease or disorder of the subject, based at least in part on processing the first document corpus with the second document corpus.

In some embodiments, (i) comprises receiving, from a remote server, the first information relating to the set of diseases or disorders encompassing the medical domain. In some embodiments, (ii) comprises receiving, from a remote server, the second information relating to the disease or disorder of the subject.

In some embodiments, the disease or disorder is cancer. In some embodiments, the cancer is selected from the group consisting of breast cancer, colorectal cancer, brain cancer, leukemia, lung cancer, skin cancer, liver cancer, pancreatic cancer, lymphoma, esophageal cancer, and cervical cancer.

In some embodiments, the first information relating to the set of diseases or disorders comprises clinical trial information, a tumor board discussion, a case summary or report, and/or outcomes reported by subjects. In some embodiments, the second information relating to the disease or disorder of the subject comprises diagnosis, stage and grade of disease, medications, vitals, laboratory results, clinical trial information, tumor board discussions, a case summary or report, and/or an outcome reported by the subject. In some embodiments, the clinical trial information is received from a clinical trial database. In some embodiments, the clinical trial database comprises a National Clinical Trial repository. In some embodiments, the clinical trial information comprises at least one of clinical trials for specific treatments for the disease or disorder, information about trial arms, information about control arms, and inclusion or exclusion criteria for clinical trials. In some embodiments, the tumor board discussion comprises information relating to at least one of tradeoffs, inclusion or exclusion criteria, and efficacy for a plurality of candidate treatments. In some embodiments, the tumor board discussion is a virtual tumor board discussion. In some embodiments, the clinical information of the subject comprises a case summary of the disease or disorder of the subject.

In some embodiments, the case summary is prepared by a health care provider of the subject. In some embodiments, the health care provider comprises a physician. In some embodiments, the physician comprises an oncologist. In some embodiments, the case summary comprises structured data, unstructured data, or a combination thereof. In some embodiments, the case summary is conveyed from an electronic health record system. In some embodiments, the case summary comprises at least one of genomic features of the subject, treatment options for the subject, and tumor load of the subject.

In some embodiments, (a) further comprises parsing the structured information or textual information of the first information according to an ontology of treatment questions. In some embodiments, the ontology comprises at least one of subject features, disease state, and types of treatments. In some embodiments, (b) further comprises parsing the structured information or textual information of the second information according to an ontology of treatment concepts. In some embodiments, the ontology comprises at least one of concepts of the subject, disease state, and types of treatments.

In some embodiments, (a) further comprises parsing the structured information or textual information of the first information to discover concepts pertaining to at least one topic selected from clinical trial information, a tumor board discussion, a case summary or report, and outcomes reported subjects. In some embodiments, (b) further comprises parsing the structured information or textual information of the second information to discover concepts pertaining to at least one topic selected from diagnosis, stage and grade of disease, medications, vitals, laboratory results, clinical trial information, a tumor board discussion, a case summary or report, and an outcome reported by the subject.

In some embodiments, (a) further comprises generating a topic space for documents received from the first set of distinct sources. In some embodiments, the topic space comprises a plurality of hierarchical topic spaces. In some embodiments, the topic space is associated with a disease state or a treatment for the disease state. In some embodiments, (b) further comprises generating a topic space for documents received from the second set of distinct sources. In some embodiments, the topic space comprises a plurality of hierarchical topic spaces. In some embodiments, the topic space is associated with a disease state or a treatment for the disease state.

In some embodiments, (a) further comprises associating a topic with a specific document received from a distinct source of the first set of distinct sources. In some embodiments, (b) further comprises associating a topic with a specific document received from a distinct source of the second set of distinct sources.

In some embodiments, (a) further comprises parsing the structured information or textual information of the first information using one or more algorithms selected from the group consisting of a text recognition algorithm, a regular expressions algorithm, a pattern recognition algorithm, an imaging recognition algorithm, a natural language processing algorithm, an optical character recognition algorithm, a term frequency-inverse document frequency (TF-IDF) algorithm, and a bag-of-words algorithm. In some embodiments, (b) further comprises parsing the structured information or textual information of the second information using one or more algorithms selected from the group consisting of a text recognition algorithm, a regular expressions algorithm, a pattern recognition algorithm, an imaging recognition algorithm, a natural language processing algorithm, an optical character recognition algorithm, a term frequency-inverse document frequency (TF-IDF) algorithm, and a bag-of-words algorithm.

In some embodiments, (a) further comprises determining, based at least in part on the parsing in (a), whether the structured information or textual information of the first information corresponds to a clinical trials database, a clinical trial arm description, a genomics database, a clinical care guideline document, a case series document, a drug database, an imaging report, a pathology report, a clinic note, a progress note, a genomics report, a laboratory test report, a diagnostic report, or a prognostic report. In some embodiments, (b) further comprises determining, based at least in part on the parsing in (b), whether the structured information or textual information of the second information corresponds to an imaging report, a pathology report, a clinic note, a progress note, a genomics report, a laboratory test report, a diagnostic report, or a prognostic report.

In some embodiments, parsing the structured information or textual information of the first information comprises at least one of case converting the structured information or textual information of the first information, removing special characters or stop words from the structured information or textual information of the first information, tokenizing the structured information or textual information of the first information, and parsing the structured information or textual information of the first information using a parser. In some embodiments, parsing the structured information or textual information of the second information comprises at least one of case converting the structured information or textual information of the second information, removing special characters or stop words from the structured information or textual information of the second information, tokenizing the structured information or textual information of the second information, and parsing the structured information or textual information of the second information using a parser.

In some embodiments, parsing the structured information or textual information of the first information comprises filtering the structured information or textual information of the first information for a disease state, a treatment for the disease state, or clinical trials associated with the disease state or the treatment for the disease state. In some embodiments, parsing the structured information or textual information of the second information comprises filtering the structured information or textual information of the second information for a disease state, a treatment for the disease state, or clinical trials associated with the disease state or the treatment for the disease state.

In some embodiments, parsing the structured information or textual information of the first information comprises extracting and standardizing inclusion or exclusion criteria. In some embodiments, parsing the structured information or textual information of the second information comprises extracting and standardizing inclusion or exclusion criteria.

In some embodiments, parsing the structured information or textual information of the first information comprises labeling the structured information or textual information of the first information with labels. In some embodiments, the labels comprise information pertaining to a disease, a treatment, an inclusion, or an exclusion. In some embodiments, parsing the structured information or textual information of the second information comprises labeling the structured information or textual information of the second information with labels. In some embodiments, the labels comprise information pertaining to a disease, a treatment, an inclusion, or an exclusion.

In some embodiments, parsing the structured information or textual information of the first information comprises performing named entity recognition. In some embodiments, performing the named entity recognition comprises at least one of ontology mapping, speech tagging, and entity type tagging. In some embodiments, parsing the structured information or textual information of the second information comprises performing named entity recognition. In some embodiments, performing the named entity recognition comprises at least one of ontology mapping, speech tagging, and entity type tagging.

In some embodiments, (a) further comprises generating a set of sub-corpuses from the first document corpus. In some embodiments, (b) further comprises generating a set of sub-corpuses from the second document corpus.

In some embodiments, (a) further comprises performing topic modeling. In some embodiments, the topic modeling in (a) comprises use of at least one of Biterm Topic Modeling (BTM), Latent Dirichlet Allocation (LDA), and Term Frequency-Inverse Document Frequency (TF-IDF) analysis. In some embodiments, the topic modeling in (a) comprises use of the LDA or TF-IDF analysis. In some embodiments, the topic modeling in (a) comprises using the topic modeling to generate ngrams of frequently occurring word combinations in the first information. In some embodiments, the frequently occurring word combinations comprise single words, word pairs, triplets, or a combination thereof. In some embodiments, the ngrams comprise a frequency of occurrence of the frequently occurring word combinations. In some embodiments, the topic modeling in (a) comprises partitioning the first document corpus into a set of topics or subtopics. In some embodiments, the partitioning comprise use of a hyperparameter. In some embodiments, the hyperparameter is received from a human user. In some embodiments, the topic modeling in (a) comprises associating relationships between ngrams and treatments, ngrams and disease state, ngrams and treatment rationales, or a combination thereof. In some embodiments, associating the relationships comprises applying a chain rule analysis to account for interaction terms. In some embodiments, the chain rule analysis comprises performing matrix multiplication.

In some embodiments, (c) further comprises mapping the ngrams of at least one of the first information and the second information to a set of candidate treatments, and generating the ranked set of candidate treatments based at least in part on the mapping. In some embodiments, the mapping comprises partitioning at least one of the first document corpus and the second document corpus based on a topic. In some embodiments, the mapping comprises computing a weight matrix, and generating the ranked set of candidate treatments based at least in part on the weight matrix. In some embodiments, the mapping comprises use of a similarity matrix to account for at least partial mismatches. In some embodiments, the mapping comprises performing matrix multiplication using the similarity matrix. In some embodiments, the similarity matrix comprises a treatment similarity matrix comprising component metrics indicative of pairwise overlap between candidate treatments in a clinical trial, evaluated over a space of a plurality of clinical trials. In some embodiments, the component metrics comprise a member selected from the group consisting of Jaccard similarity between candidate treatments, cosine similarity between candidate treatments, Jaro-Winkler (J-W) distance between candidate treatments, and Jaccard syllable similarity between candidate treatments. In some embodiments, the component metrics comprise at least two members selected from the group consisting of Jaccard similarity between candidate treatments, cosine similarity between candidate treatments, Jaro-Winkler (J-W) distance between candidate treatments, and Jaccard syllable similarity between candidate treatments. In some embodiments, the one or more computer processors are individually or collectively programmed to further calculate an ensemble score for at least two treatment similarity matrices. In some embodiments, calculating the ensemble score comprises performing a dimensionality analysis. In some embodiments, the dimensionality analysis is selected from the group consisting of principal component analysis (PCA), t-distributed stochastic neighbor embedding (t-SNE), and uniform manifold approximation and projection (UMAP), and human supervision. In some embodiments, the similarity matrix comprises a disease similarity matrix comprising component metrics indicative of pairwise overlap between diseases in a clinical trial, evaluated over a space of a plurality of clinical trials. In some embodiments, the component metrics comprise a member selected from the group consisting of Jaccard similarity between diseases, cosine similarity between diseases, Jaro-Winkler (J-W) distance between diseases, and Jaccard syllable similarity between diseases. In some embodiments, the component metrics comprise at least two members selected from the group consisting of Jaccard similarity between diseases, cosine similarity between diseases, Jaro-Winkler (J-W) distance between diseases, and Jaccard syllable similarity between diseases. In some embodiments, the one or more computer processors are individually or collectively programmed to further calculate an ensemble score for at least two disease similarity matrices. In some embodiments, calculating the ensemble score comprises performing a dimensionality analysis. In some embodiments, the dimensionality analysis is selected from the group consisting of principal component analysis (PCA), t-distributed stochastic neighbor embedding (t-SNE), and uniform manifold approximation and projection (UMAP), and human supervision. In some embodiments, the mapping comprises using latent semantic analysis. In some embodiments, the mapping comprises performing a plurality of mappings comprising at least a first mapping from the ngrams to a topic, subtopic, or disease, and a second mapping from the topic, the subtopic, or the disease to the set of candidate treatments.

In some embodiments, (c) further comprises combining outputs from a plurality of mappings, and generating the ranked set of candidate treatments based at least in part on the combined outputs. In some embodiments, combining the outputs comprises summing the outputs from the plurality of mappings. In some embodiments, combining the outputs comprises using a set of weights to calculate a weighted sum of the outputs from the plurality of mappings. In some embodiments, combining the outputs comprises normalizing or scaling the set of weights. In some embodiments, the set of weights comprises values between 0 and 1. In some embodiments, the set of weights is adjusted using a training set. In some embodiments, the set of weights is adjusted by XGBoost, Bayesian rejection sampling, Thompson Sampling, upper confidence bound sampling, or knowledge gradient sampling. In some embodiments, the set of weights is adjusted based on a distance metric between a model-predicted treatment ranking and an observed treatment ranking. In some embodiments, the distance metric comprises a Kendall tau distance.

In some embodiments, processing the first document corpus with the second document corpus in (c) comprises comparing the first document corpus and second document corpus to each other.

In some embodiments, the one or more computer processors are individually or collectively programmed to further perform at least one iteration of (i) and (a) to incorporate new or updated medical information into the first document corpus. In some embodiments, (a) comprises using a Bayesian update process to incorporate the new or updated medical information into the first document corpus. In some embodiments, (a) comprises, subsequent to the subject being followed to a specified endpoint, incorporating the new or updated medical information of the subject into the first document corpus, thereby allowing additional subjects to benefit therefrom. In some embodiments, the one or more computer processors are individually or collectively programmed to further perform (ii), (b), and (c) for an additional subject in need of an individual recommendation for medical treatment.

In another aspect, the present disclosure provides a non-transitory computer-readable medium comprising machine-executable code that, upon execution by one or more computer processors, implements a method for generating an individual recommendation for medical treatment of a subject, the method comprising: (a) receiving, from a first set of distinct sources, first information relating to a set of diseases or disorders encompassing a medical domain; (b) processing the first information relating to the set of diseases or disorders to generate a first document corpus, wherein processing the first information comprises parsing structured information or textual information of the first information; (c) receiving, from a second set of distinct sources, second information relating to a disease or disorder of the subject, wherein the second information comprises a clinical information of the subject; (d) processing the second information relating to the disease or disorder of the subject to generate a second document corpus, wherein processing the second information comprises parsing structured information or textual information of the second information; and (e) generating a ranked set of candidate treatments for treating the disease or disorder of the subject, based at least in part on processing the first document corpus with the second document corpus.

In some embodiments, (a) comprises receiving, from a remote server, the first information relating to the set of diseases or disorders encompassing the medical domain. In some embodiments, (c) comprises receiving, from a remote server, the second information relating to the disease or disorder of the subject.

In some embodiments, the disease or disorder is cancer. In some embodiments, the cancer is selected from the group consisting of breast cancer, colorectal cancer, brain cancer, leukemia, lung cancer, skin cancer, liver cancer, pancreatic cancer, lymphoma, esophageal cancer, and cervical cancer.

In some embodiments, the first information relating to the set of diseases or disorders comprises clinical trial information, a tumor board discussion, a case summary or report, and/or outcomes reported by subjects. In some embodiments, the second information relating to the disease or disorder of the subject comprises diagnosis, stage and grade of disease, medications, vitals, laboratory results, clinical trial information, tumor board discussions, a case summary or report, and/or an outcome reported by the subject. In some embodiments, the clinical trial information is received from a clinical trial database. In some embodiments, the clinical trial database comprises a National Clinical Trial repository. In some embodiments, the clinical trial information comprises at least one of clinical trials for specific treatments for the disease or disorder, information about trial arms, information about control arms, and inclusion or exclusion criteria for clinical trials. In some embodiments, the tumor board discussion comprises information relating to at least one of tradeoffs, inclusion or exclusion criteria, and efficacy for a plurality of candidate treatments. In some embodiments, the tumor board discussion is a virtual tumor board discussion. In some embodiments, the clinical information of the subject comprises a case summary of the disease or disorder of the subject.

In some embodiments, the case summary is prepared by a health care provider of the subject. In some embodiments, the health care provider comprises a physician. In some embodiments, the physician comprises an oncologist. In some embodiments, the case summary comprises structured data, unstructured data, or a combination thereof. In some embodiments, the case summary is conveyed from an electronic health record system. In some embodiments, the case summary comprises at least one of genomic features of the subject, treatment options for the subject, and tumor load of the subject.

In some embodiments, (b) further comprises parsing the structured information or textual information of the first information according to an ontology of treatment questions. In some embodiments, the ontology comprises at least one of subject features, disease state, and types of treatments. In some embodiments, (d) further comprises parsing the structured information or textual information of the second information according to an ontology of treatment concepts. In some embodiments, the ontology comprises at least one of concepts of the subject, disease state, and types of treatments.

In some embodiments, (b) further comprises parsing the structured information or textual information of the first information to discover concepts pertaining to at least one topic selected from clinical trial information, a tumor board discussion, a case summary or report, and outcomes reported subjects. In some embodiments, (d) further comprises parsing the structured information or textual information of the second information to discover concepts pertaining to at least one topic selected from diagnosis, stage and grade of disease, medications, vitals, laboratory results, clinical trial information, a tumor board discussion, a case summary or report, and an outcome reported by the subject.

In some embodiments, (b) further comprises generating a topic space for documents received from the first set of distinct sources. In some embodiments, the topic space comprises a plurality of hierarchical topic spaces. In some embodiments, the topic space is associated with a disease state or a treatment for the disease state. In some embodiments, (d) further comprises generating a topic space for documents received from the second set of distinct sources. In some embodiments, the topic space comprises a plurality of hierarchical topic spaces. In some embodiments, the topic space is associated with a disease state or a treatment for the disease state.

In some embodiments, (b) further comprises associating a topic with a specific document received from a distinct source of the first set of distinct sources. In some embodiments, (d) further comprises associating a topic with a specific document received from a distinct source of the second set of distinct sources.

In some embodiments, (b) further comprises parsing the structured information or textual information of the first information using one or more algorithms selected from the group consisting of a text recognition algorithm, a regular expressions algorithm, a pattern recognition algorithm, an imaging recognition algorithm, a natural language processing algorithm, an optical character recognition algorithm, a term frequency-inverse document frequency (TF-IDF) algorithm, and a bag-of-words algorithm. In some embodiments, (d) further comprises parsing the structured information or textual information of the second information using one or more algorithms selected from the group consisting of a text recognition algorithm, a regular expressions algorithm, a pattern recognition algorithm, an imaging recognition algorithm, a natural language processing algorithm, an optical character recognition algorithm, a term frequency-inverse document frequency (TF-IDF) algorithm, and a bag-of-words algorithm.

In some embodiments, (b) further comprises determining, based at least in part on the parsing in (b), whether the structured information or textual information of the first information corresponds to a clinical trials database, a clinical trial arm description, a genomics database, a clinical care guideline document, a case series document, a drug database, an imaging report, a pathology report, a clinic note, a progress note, a genomics report, a laboratory test report, a diagnostic report, or a prognostic report. In some embodiments, (d) further comprises determining, based at least in part on the parsing in (d), whether the structured information or textual information of the second information corresponds to an imaging report, a pathology report, a clinic note, a progress note, a genomics report, a laboratory test report, a diagnostic report, or a prognostic report.

In some embodiments, parsing the structured information or textual information of the first information comprises at least one of case converting the structured information or textual information of the first information, removing special characters or stop words from the structured information or textual information of the first information, tokenizing the structured information or textual information of the first information, and parsing the structured information or textual information of the first information using a parser. In some embodiments, parsing the structured information or textual information of the second information comprises at least one of case converting the structured information or textual information of the second information, removing special characters or stop words from the structured information or textual information of the second information, tokenizing the structured information or textual information of the second information, and parsing the structured information or textual information of the second information using a parser.

In some embodiments, parsing the structured information or textual information of the first information comprises filtering the structured information or textual information of the first information for a disease state, a treatment for the disease state, or clinical trials associated with the disease state or the treatment for the disease state. In some embodiments, parsing the structured information or textual information of the second information comprises filtering the structured information or textual information of the second information for a disease state, a treatment for the disease state, or clinical trials associated with the disease state or the treatment for the disease state.

In some embodiments, parsing the structured information or textual information of the first information comprises extracting and standardizing inclusion or exclusion criteria. In some embodiments, parsing the structured information or textual information of the second information comprises extracting and standardizing inclusion or exclusion criteria.

In some embodiments, parsing the structured information or textual information of the first information comprises labeling the structured information or textual information of the first information with labels. In some embodiments, the labels comprise information pertaining to a disease, a treatment, an inclusion, or an exclusion. In some embodiments, parsing the structured information or textual information of the second information comprises labeling the structured information or textual information of the second information with labels. In some embodiments, the labels comprise information pertaining to a disease, a treatment, an inclusion, or an exclusion.

In some embodiments, parsing the structured information or textual information of the first information comprises performing named entity recognition. In some embodiments, performing the named entity recognition comprises at least one of ontology mapping, speech tagging, and entity type tagging. In some embodiments, parsing the structured information or textual information of the second information comprises performing named entity recognition. In some embodiments, performing the named entity recognition comprises at least one of ontology mapping, speech tagging, and entity type tagging.

In some embodiments, (b) further comprises generating a set of sub-corpuses from the first document corpus. In some embodiments, (d) further comprises generating a set of sub-corpuses from the second document corpus.

In some embodiments, (b) further comprises performing topic modeling. In some embodiments, the topic modeling in (b) comprises use of at least one of Biterm Topic Modeling (BTM), Latent Dirichlet Allocation (LDA), and Term Frequency-Inverse Document Frequency (TF-IDF) analysis. In some embodiments, the topic modeling in (b) comprises use of the LDA or TF-IDF analysis. In some embodiments, the topic modeling in (b) comprises using the topic modeling to generate ngrams of frequently occurring word combinations in the first information. In some embodiments, the frequently occurring word combinations comprise single words, word pairs, triplets, or a combination thereof. In some embodiments, the ngrams comprise a frequency of occurrence of the frequently occurring word combinations. In some embodiments, the topic modeling in (b) comprises partitioning the first document corpus into a set of topics or subtopics. In some embodiments, the partitioning comprise use of a hyperparameter. In some embodiments, the hyperparameter is received from a human user. In some embodiments, the topic modeling in (b) comprises associating relationships between ngrams and treatments, ngrams and disease state, ngrams and treatment rationales, or a combination thereof. In some embodiments, associating the relationships comprises applying a chain rule analysis to account for interaction terms. In some embodiments, the chain rule analysis comprises performing matrix multiplication.

In some embodiments, (e) further comprises mapping the ngrams of at least one of the first information and the second information to a set of candidate treatments, and generating the ranked set of candidate treatments based at least in part on the mapping. In some embodiments, the mapping comprises partitioning at least one of the first document corpus and the second document corpus based on a topic. In some embodiments, the mapping comprises computing a weight matrix, and generating the ranked set of candidate treatments based at least in part on the weight matrix. In some embodiments, the mapping comprises use of a similarity matrix to account for at least partial mismatches. In some embodiments, the mapping comprises performing matrix multiplication using the similarity matrix. In some embodiments, the similarity matrix comprises a treatment similarity matrix comprising component metrics indicative of pairwise overlap between candidate treatments in a clinical trial, evaluated over a space of a plurality of clinical trials. In some embodiments, the component metrics comprise a member selected from the group consisting of Jaccard similarity between candidate treatments, cosine similarity between candidate treatments, Jaro-Winkler (J-W) distance between candidate treatments, and Jaccard syllable similarity between candidate treatments. In some embodiments, the component metrics comprise at least two members selected from the group consisting of Jaccard similarity between candidate treatments, cosine similarity between candidate treatments, Jaro-Winkler (J-W) distance between candidate treatments, and Jaccard syllable similarity between candidate treatments. In some embodiments, the method further comprises calculating an ensemble score for at least two treatment similarity matrices. In some embodiments, calculating the ensemble score comprises performing a dimensionality analysis. In some embodiments, the dimensionality analysis is selected from the group consisting of principal component analysis (PCA), t-distributed stochastic neighbor embedding (t-SNE), and uniform manifold approximation and projection (UMAP), and human supervision. In some embodiments, the similarity matrix comprises a disease similarity matrix comprising component metrics indicative of pairwise overlap between diseases in a clinical trial, evaluated over a space of a plurality of clinical trials. In some embodiments, the component metrics comprise a member selected from the group consisting of Jaccard similarity between diseases, cosine similarity between diseases, Jaro-Winkler (J-W) distance between diseases, and Jaccard syllable similarity between diseases. In some embodiments, the component metrics comprise at least two members selected from the group consisting of Jaccard similarity between diseases, cosine similarity between diseases, Jaro-Winkler (J-W) distance between diseases, and Jaccard syllable similarity between diseases. In some embodiments, the method further comprises calculating an ensemble score for at least two disease similarity matrices. In some embodiments, calculating the ensemble score comprises performing a dimensionality analysis. In some embodiments, the dimensionality analysis is selected from the group consisting of principal component analysis (PCA), t-distributed stochastic neighbor embedding (t-SNE), and uniform manifold approximation and projection (UMAP), and human supervision. In some embodiments, the mapping comprises using latent semantic analysis. In some embodiments, the mapping comprises performing a plurality of mappings comprising at least a first mapping from the ngrams to a topic, subtopic, or disease, and a second mapping from the topic, the subtopic, or the disease to the set of candidate treatments.

In some embodiments, (e) further comprises combining outputs from a plurality of mappings, and generating the ranked set of candidate treatments based at least in part on the combined outputs. In some embodiments, combining the outputs comprises summing the outputs from the plurality of mappings. In some embodiments, combining the outputs comprises using a set of weights to calculate a weighted sum of the outputs from the plurality of mappings. In some embodiments, combining the outputs comprises normalizing or scaling the set of weights. In some embodiments, the set of weights comprises values between 0 and 1. In some embodiments, the set of weights is adjusted using a training set. In some embodiments, the set of weights is adjusted by XGBoost, Bayesian rejection sampling, Thompson Sampling, upper confidence bound sampling, or knowledge gradient sampling. In some embodiments, the set of weights is adjusted based on a distance metric between a model-predicted treatment ranking and an observed treatment ranking. In some embodiments, the distance metric comprises a Kendall tau distance.

In some embodiments, processing the first document corpus with the second document corpus in (e) comprises comparing the first document corpus and second document corpus to each other.

In some embodiments, the method further comprises performing at least one iteration of (a) and (b) to incorporate new or updated medical information into the first document corpus. In some embodiments, (b) comprises using a Bayesian update process to incorporate the new or updated medical information into the first document corpus. In some embodiments, (b) comprises, subsequent to the subject being followed to a specified endpoint, incorporating the new or updated medical information of the subject into the first document corpus, thereby allowing additional subjects to benefit therefrom. In some embodiments, the method further comprises performing (c) to (e) for an additional subject in need of an individual recommendation for medical treatment.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein) of which:

FIG. 1 depicts an example of a page from the NCCN Guidelines for treating metastatic pancreatic cancer.

FIG. 2 is a screenshot showing an example of a case summary for a patient with a brain tumor, along with the treatment options selected by a system of the present disclosure.

FIG. 3 shows an example of the high-level data flow of the training portion of an embodiment.

FIG. 4 shows the domain-specific data ingestor 311 of FIG. 3 in more detail.

FIG. 5 shows the domain-specific data ingestor 312 of FIG. 3 in more detail.

FIG. 6A shows an example of the word frequency for a topic identified in a document corpus.

FIG. 6B illustrates an example of a graph of ngrams extracted from an entire document corpus.

FIG. 7A diagrams an example of the process flow for an embodiment of the mapper “Ngram-to-Drug.

FIG. 7B diagrams an example of the process flow for an embodiment of the mapper “Ngram-to-Drug.

FIG. 7C illustrates an example of a portion of the table used to derive the treatment similarity matrix 715 depicted in FIG. 7B.

FIG. 8 provides an example of using the Latent Semantic Analysis module to create subtopics.

FIG. 9 diagrams an example of the process flow for the mapper “Ngram-to-Topic-to-Drug.

FIG. 10A diagrams an example of the process flow for one embodiment of the mapper “Ngram-to-Disease-to-Drug.

FIG. 10B diagrams an example of the process flow for an embodiment of the mapper “Ngram-to-Disease-to-Drug.

FIG. 10C illustrates an example of a portion of the table used to derive the disease similarity matrix 1015 depicted in FIG. 10B.

FIG. 11 illustrates an example of the Ngram-to-Drug-Ranks Engine.

FIG. 12 illustrates an example of optimizing a weighting vector using machine learning.

FIG. 13 shows an example of a runtime environment in the context of a patient case summary.

FIG. 14 illustrates a computer system programmed to implement methods and systems of the present disclosure.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

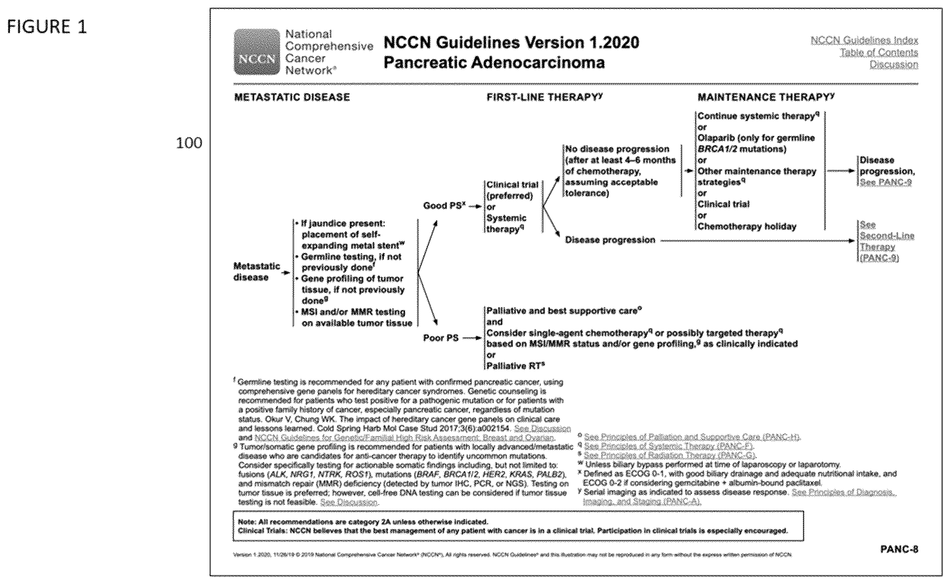

In many clinically delineated stages of disease, there are well established clinical guidelines. For example, the National Comprehensive Cancer Network (NCCN) publishes detailed flowcharts for disease state for most major type of cancer every one to three years, based upon accumulated evidence from published clinical trials and abstracts managed by a team of experts. FIG. 1 depicts an example of one such page 100, covering the metastatic stage of pancreatic cancer. This flowchart bifurcates on the performance status (PS) of the patient, so that patients who meet a minimum qualitative level may receive either a clinical trial or systemic chemotherapy, and those who don't may receive palliative care.

For patients with a good performance status, a clinical trial (difficult to enroll in, with highly variable and unpredictable outcome) may be preferred over the standard of care (systemic chemotherapy), meaning that the standard of care outcome is widely acknowledged to be dire. Furthermore, in cancer, only about 5% of patients who exhaust the standard of care may be ever successfully enrolled in a clinical trial, owing to the trial-specific inclusion and exclusion criteria, being too distant from the site of a clinical trial, or other reasons.

There may be a third alternative available to physicians, which is to prescribe therapies off-label and/or prescribe expanded access drugs, alone or in combination, without their patients needing to travel to a clinical trial site. This can be done directly by the physician, or by the physician and patient participating in a decentralized trial. A physician can access these potential combination therapies via the system of the present disclosure.

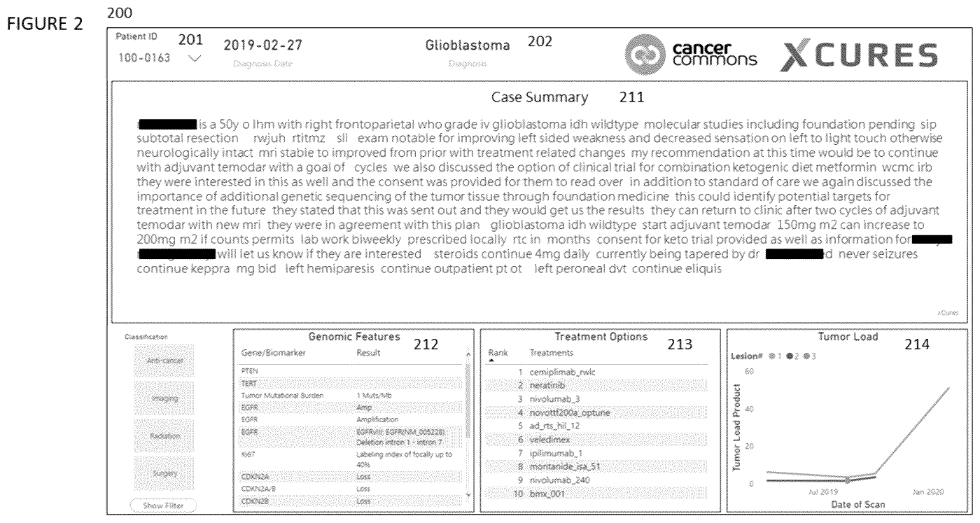

FIG. 2 shows an example of a screenshot from the system 200, where a physician has entered patient data into the system, creating a case summary 211 (with some personal information redacted). The general diagnosis is shown above 202, and the physician can navigate to other information panes in the system via dropdown menu 201. In the lower part of the window are smaller panes showing genomic features 212, treatment options 213, and tumor load 214.

The treatment options 213 shown here may be automatically generated from case summary 211, and may be ranked. For example, the ranking may be done such that item ranked 1 on the list, cemiplimab, is the most highly recommended option, and the last item on the list, bmx_001, is the least recommended option on the list (which may not be a bad option, but rather 10th out of list of 10 good options).

Generating these options may comprise a number of operations. First, sources of reliable, trusted knowledge may be ingested to provide a document corpus that may serve as reference material. Then, this reference material may be organized according to the questions that may be asked. That is, the ontology of the questions (patient features, disease state, types of treatments, etc.) may be properly scoped.

There may be two phases to this process: a training phase, and the execution phase. The training phase may comprise the analysis of large amounts of data from a variety of sources to perform a variety of tasks, such as:
Discover concepts in documents pertaining to clinical trials, tumor board discussions regarding specific patients, and other such source materials;
Generate a topic space for a corpus of documents; and,
Associate one or more topics with specific documents

There can be multiple topic spaces associated with a corpus of documents, and these may be hierarchical. For example, it may be necessary to extract the disease state. A topic may be “autoimmune disease,” with a subtopic of “history of autoimmune disease” or “systemic corticosteroid therapy.” It may also be necessary to extract the drugs associated with that disease state, such as “prednisone.”

While the case summary 211 is depicted in this embodiment of the present disclosure as a textual description of the patient's status and history, in general the case summary (or for that matter, any type of document methods and systems of the present disclosure can intake) may be a mix of structured and unstructured data. In particular, a patient's status may be conveyed from an Electronic Health Record (EHR) System via any number of formats, such as HL7 or FHIR, which may make reference to specific codings and ontologies such as LOINC, SNOMED CT, and others. Other interchange formats for structured data may include JSON format and XML.

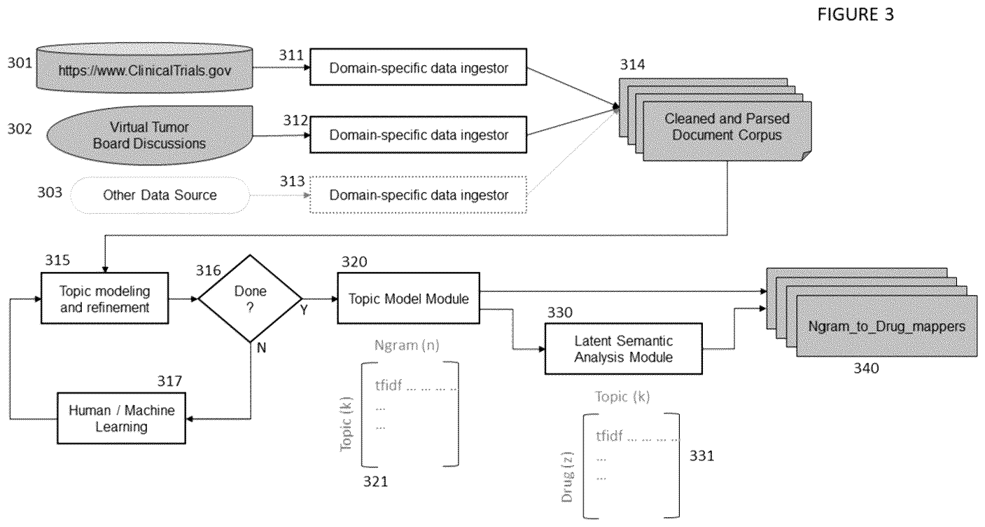

FIG. 3 depicts an example of operations performed to accomplish this automatic ranking, in the form of the high-level data flow of an embodiment of the present disclosure. Two data sources are shown. The system may read clinical trial data from the National Clinical Trial repository at www.ClinicalTrials.gov 301 and then feed that data into a domain-specific data ingestor 311, which performs a number of tasks, to be described shortly, to output cleaned and parsed documents from www.ClinicalTrials.gov describing each trial. These documents may refer to trials of specific treatments for diseases, describing trial arms, control arms, inclusion and exclusion criteria, etc., and thus may have a wealth of information about how and when experimental treatments should and should not be used.

Similarly, a slightly different domain-specific data ingestor 312 may take data from virtual tumor board discussions 302 (textual data—emails, SMS, voice-to-text, etc.) and convert it to cleaned and parsed documents. The virtual tumor board discussions may relate to individual patient cases, and discuss the tradeoffs of using specific treatment regimens, usually in the context of choosing from a set of four to eight possible treatment regimens. Thus, they may contain information about inclusion and exclusion criteria (e.g., “does the patient have excessive edema?”), relative ranking information about expert-perceived treatment efficacy, and expert's rules of thumb (e.g., “don't use class X drugs after partial resections of type Y tumors”).

Since the discussions and data sources 301 and 302 may be slightly different, the data ingestors 311 and 312 may be domain-specific, and may not always be identical. There may be times where one data ingestor can be used for different data sources.

The architecture of a system or method of the present disclosure allows for an arbitrary number of other data sources 303 and additional domain-specific data ingestors 313 to expand the capabilities of the system to ingest data from other relevant sources of data. For example, patient-reported outcomes surveys (PROs) may serve as an additional source of data. Additionally, every patient in an EHR system with features (diagnosis, treatment, medical commentary, etc.) and associated outcomes may have their data ingested into the system, potentially making it more intelligent over time.

The result of parsing all sources 301, and/or 302, and/or any additional sources 303 of data, through the ingestors, may be a corpus of cleaned and parsed documents 314.

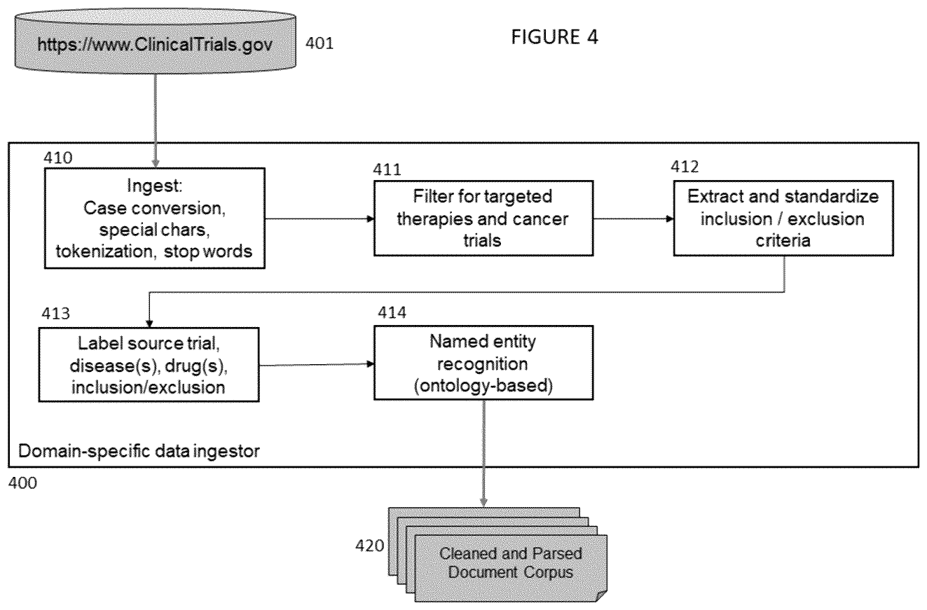

The ingestors are now discussed. In this section, it may be assumed for illustrative purposes that this tool is being used for cancer. An example of the domain-specific data ingestor 311 of FIG. 3 is shown in more detail in FIG. 4. The input to the ingestor may be the data from www.ClinicalTrials.gov 401, which first enters operation 410, where some or all of the data is case converted to a standard (e.g., all lowercase), special characters are removed, the text is tokenized, and stop words are removed. Structured data may be handled by its appropriate parser. Next, in operation 411, the text may be filtered for the specific therapies administered in that trial, as well as the cancer or cancers that are targeted. Therefore, for this application, the tool may filter out trials that apply to chronic diseases. Some trials may pertain to multiple cancers, and some trials may have multiple trial arms that use different treatments in the different arms (different drugs, or a drug in combination with other drugs, or different dosages).

In operation 412, inclusion and/or exclusion criteria, such as patient performance status, prior failed treatments, minimum and maximum allowed lab values indicating adequate organ function, etc., may be extracted and standardized. In operation 413, some or all of the prior data may be labeled (e.g., disease, drugs, inclusion and/or exclusion) in the text. In operation 414, named entity recognition is performed. This may be done via a combination of standard ontologies (such as the National Cancer Institute Thesaurus) plus custom additions to account for the fact that no existing ontology may be quite adequate for this task. In some embodiments, named entity recognition may comprise part of speech tagging and entity type tagging, activities which may not be considered in some approaches for ontology mapping. The result may be cleaned and parsed text may be outputted to form part of the document corpus 420.

Again, while this example has been tailored for the domain of cancer, the methods and systems of the present disclosure may be used for other domains as well, such as chronic diseases.

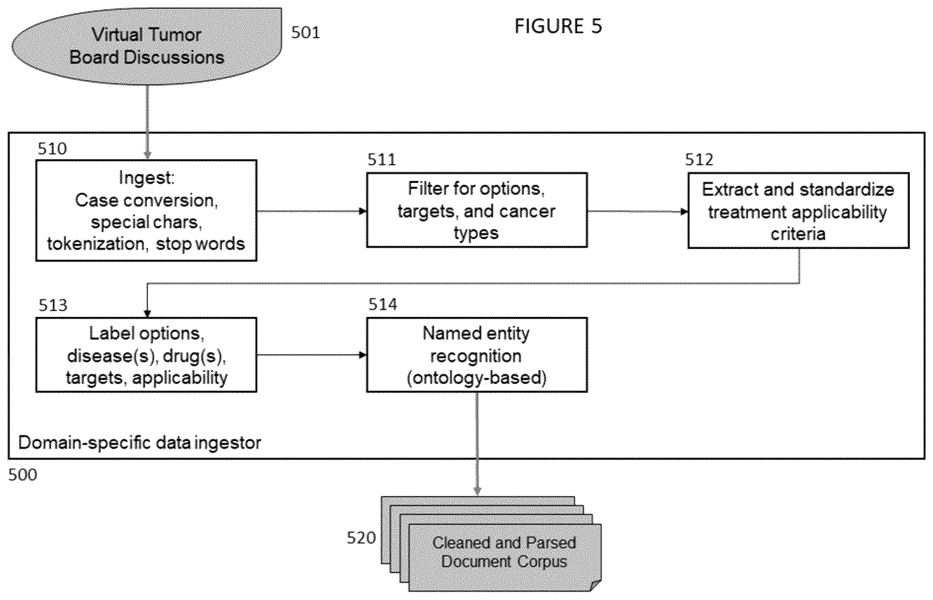

Another example of the domain-specific data ingestor 311 of FIG. 3 is shown in more detail in FIG. 5, with the virtual tumor board discussion 501 feeding into operation 510, where some or all of the data may be case converted to a standard (e.g., all lowercase), special characters may be removed, the text may be tokenized, and stop words may be removed. Structured data may be handled by its appropriate parser. Operation 511 may be slightly different, because instead of looking at different trial arms, the system may be looking at a tumor board in which experts are discussing, e.g., four to eight options for a single cancer for one patient. The document corpus for all tumor boards may cover many cancers; therefore, sub-corpuses can be created for a single cancer, and topic models can be developed accordingly. Operation 512, where the extraction of treatment criteria occurs, may be based not on trial criteria, but on the experts' collective wisdom and expertise. This may be more rationales-based. Operations 513 and 514 may be similar to operations 413 and 414 of FIG. 4.

Returning to FIG. 3, the next phase in the training portion of the method of the present disclosure may comprise topic modeling and refinement, shown in the loop comprising operations 315, 316, and 317. In practice, this may comprise a human interaction in the loop to overcome the “cold start” problem (e.g., starting the process of ranking items when there is no data) initially, but it can be run purely with machine learning thereafter. A number of techniques may be employed, such as:
Biterm Topic Modeling (BTM),
Latent Dirichlet Allocation (LDA), and/or
Term Frequency-Inverse Document Frequency (TF-IDF) analysis.

While all of these may be unsupervised machine learning techniques, human supervision may be performed to put meaningful labels on some classification results, so that interpretation of the results makes sense to a practitioner. This may be clearly identified in the accompanying text. BTM and LDA may be performed to partition the document corpus into a set of topics and subtopics. Human guidance may be used to select hyperparameters, such as deciding how many topics the document corpus is to be divided into, and how many subtopics per topic is sufficient.

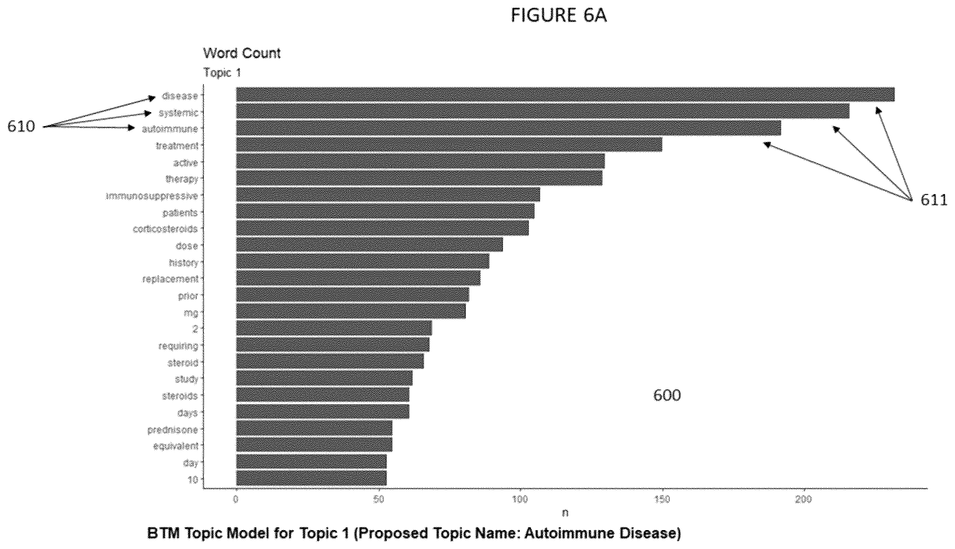

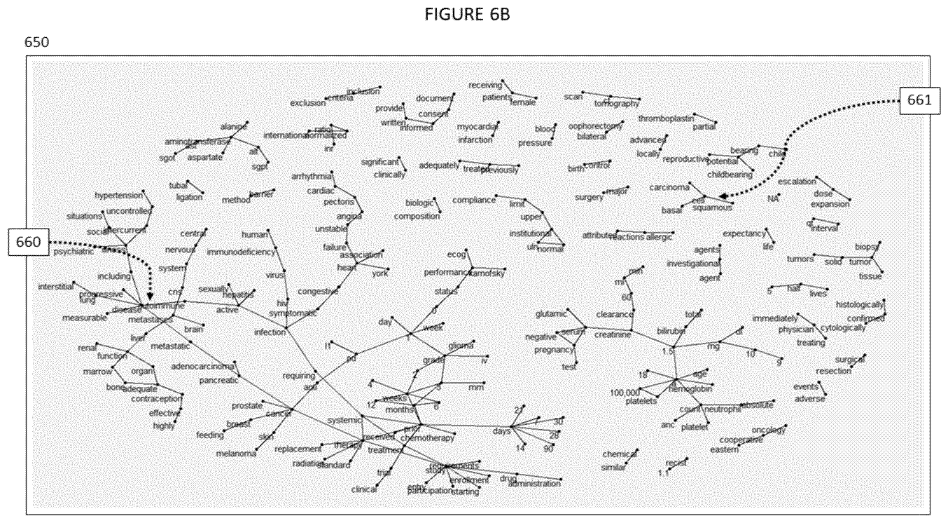

TF-IDF may be performed used to identify terms of importance that occur frequently in a document, such a patient case summary or clinical trial description, but are relatively uncommon across the corpus of documents. Ngrams of the most frequently occurring word combinations (single words, word pairs, triplets, and so forth), may also be extracted and scored, according to TF-IDF. By way of example, FIG. 6A shows an example of the word frequency for one such topic that has been identified. Graph 600 lists the top terms in descending order by frequency of occurrence in the corpus. The top words 610 are “disease,” “systemic,” and “autoimmune.” The frequency of occurrence is denoted by the length of bars 611.

Examples of ngrams extracted from the entire corpus are shown in FIG. 6B in graph 650. Label 660 points to the section in the graph where “autoimmune” and “disease” are linked, but “systemic” is not found attached to that part of the graph. Thus, “autoimmune disease” may be a reasonable name for this topic. This part of the system may be semi-automated, in that names are suggested by a computer, but a human approves and possibly alters the topic names, to ensure that the final topics are intuitive and understandable to human experts. Terms may be assigned to topics with weightings and may be associated with different weights relative to multiple topics.

Label 661, by way of another example, shows another ngram cluster from which both “squamous cell carcinoma” and “basal cell carcinoma,” closely related diseases, are derived.

Topics can relate to the relationship between ngrams and treatments, ngrams and disease state, ngrams and treatment rationales, etc. A “chain rule” analysis may apply, via matrix multiplication, wherein interaction terms may be accounted for by analyzing ngrams to disease and then disease to drug. This may be done in addition to analyzing direct relationships in the texts from ngrams to drug. These richer relationships help lead to more robust recommendations from methods and systems of the present disclosure.

Returning to FIG. 3, after the initial topic modeling is completed, flow may exit decision operation 316 at the “Y” branch, and preparation may begin for creating the runtime environment. Either or both of the Topic Model Module 320 and Latent Semantic Analysis Module 330 may be used to produce Ngram_to_Drug_mappers 340, which may be modules that contain the matrices that compute the treatment rankings.

Throughout the rest of this discussion, the term “drug” may be used as an example, but may be substituted without loss of generality with any treatment in general, including, but not limited to: pharmacological interventions, plus non-pharmacological therapies including surgery, radiation, dietary therapy, electrostimulation therapies, etc. Because of the space limitations for drawings, the term “drug” may be used for illustrative purposes. This notation may be understood to be a shorthand and is not meant to be limiting in any way.

The simplest modules for this may be the ngram-to-drug computations that link directly from the ngrams to the TF-IDF weighted values for each value in the output vector. For example, if Topic Model Module 320 is given as input “Drugs” as the topics, this may generate an ngram to drugs matrix with TF-IDF weights. Topic Model Module 320 may take as input a vector of ngrams of length n, a topic vector of length k by which to partition the document corpus, and may then compute the TF-IDF weight matrix 321, and use this to create a module, called a “mapper,” that is to be added to the list of ngram_to_drug_mappers 340.

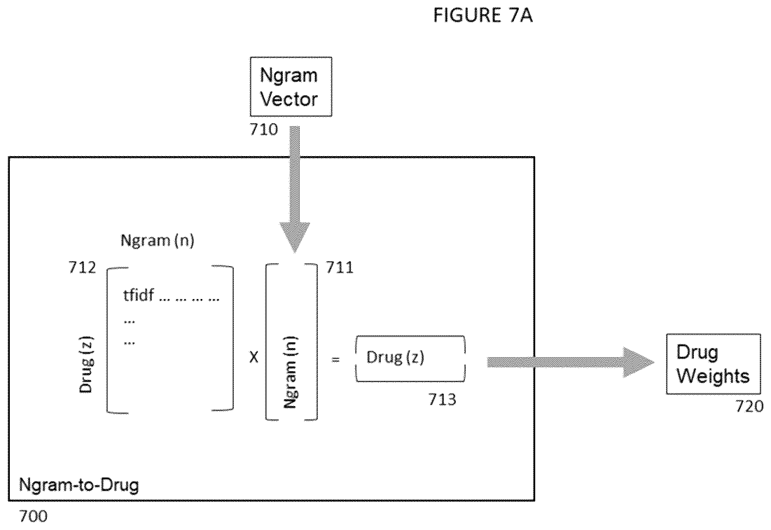

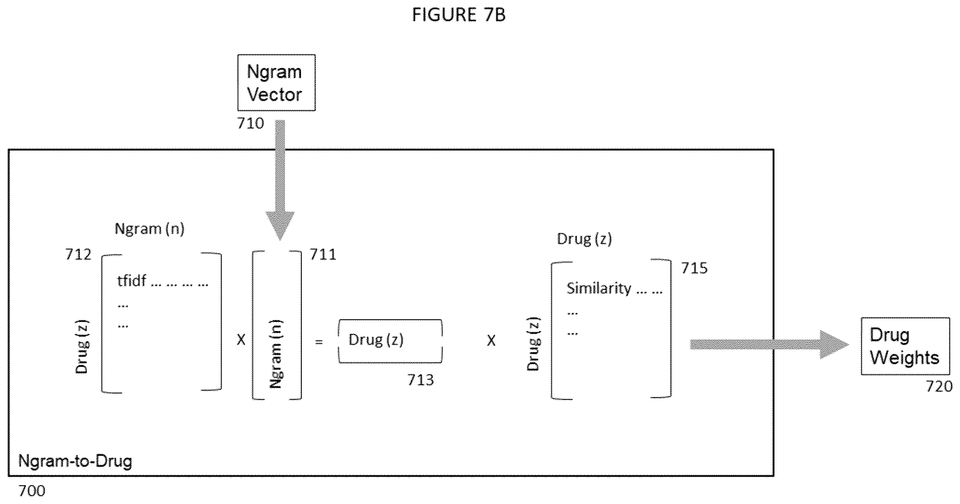

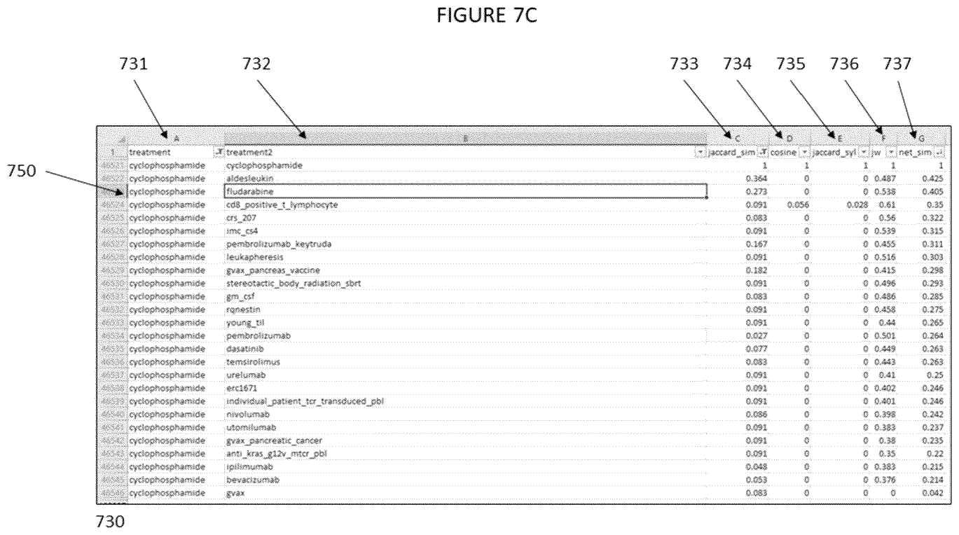

An example of such a mapper is shown in FIG. 7A for the mapping from “Ngram-to-Drug” ranking 700. In this example, the mapper 700 may take as input a vector 710 of the ngram weights for a specific document (for example, the case summary for a particular patient, such as the patient case summary 211 of FIG. 2). In this example, the ngram vector is of length n, and there are z different possible drugs. Therefore, the TF-IDF matrix 712 may be n x z in size. The input vector 710 may be coerced into the form of a column vector 711, and then TF-IDF matrix 712 may be multiplied by column vector 711 to create the drug weightings row vector of width z 713. This may be outputted from the mapper to become the output weights 720.

However, this type of mapping may not necessarily work well, because it may miss some or many potential matches, for various reasons: the case summary may be partially complete and may miss a few features of the disease state description; there may be misspellings in words; the physician may have misdiagnosed and specified a close, but related diagnosis, etc. Therefore, some embodiments employ mappers that use an additional operation of multiplication by a “similarity matrix” to account for these types of issues.

FIG. 7B illustrates an embodiment of such a mapper. It may be identical in function to that of FIG. 7A from the input Ngram Vector 710 up until the point of the drug weightings row vector 713. However, starting at this point, vector 713 may be multiplied by a square matrix of the same dimension as vector 713's length, the drug similarity matrix 715, to adjust the final weights and output the resulting output weights 720.

The drug similarity matrix 715 may be computed at least in part by calculating a number of different metrics, which affect different dimensions of similarity, and then combining them into one ensemble metric. The component metrics can include, but are not limited to, one or more of the following:
A metric of overlap between occurrence of the two drugs in a clinical trial, summed over the space of trials. This can be achieved using a number of metrics, such as Jaccard similarity.
Cosine similarity between terms defining the drug, where the cosine between two terms is the angle between the vector representation of the components of the terms, each term being a word, syllable, letter, etc., where the components (“words,” “syllables,” “letters”) comprise the dimensions of the space.
Jaccard similarity between terms defining the drug, where the cosine between two terms is the angle between the vector representation of the components of the terms, each term being a word, syllable, letter, etc., where the components (“words,” “syllables,” “letters”) comprise the dimensions of the space. Note that Jaccard similarity of the terms of the drug name may be different than Jaccard similarity of the drug usage within trials; either or both may be used.
Jaro-Winkler (J-W) distance between the terms. This metric measures string distance and helps catch misspellings, for managing typographic errors or other conventions, which are common in both clinic notes and clinical trials records. For example, consider “5fu” versus “5-fu” which are both abbreviations for the treatment 5-fluorouracil. J-W places modified weight on the first few characters of a string based on empirical observations around where in a word human beings are likely to make typographical errors. The use of multiple similarity measures may further be combined to generate ensemble scores for similarity matrices using simple averages, dimensionality analysis techniques including principal component analysis (PCA), t-distributed stochastic neighbor embedding (t-SNE), and uniform manifold approximation and projection (UMAP), and human supervision.
Jaccard syllable similarity relies on the fact that drug names encode information on their function and purpose, so that drugs that perform similar tasks—and are therefore similar—share syllables (the same principle applies to diseases). For example:
   Monoclonal antibodies end with the stem “-mab”
      Chimeric human-mouse—drugs ending in “-ximab” (i.e., rituximab)
      Humanized mouse—drugs ending in “-zumab” (i.e., bevacizumab)
      Fully human—drugs ending in “-mumab” (i.e., ipilimumab)
   Small molecule inhibitors end with the stem “-ib”
   Small molecule inhibitors of the protein BRAF include “raf” (i.e., dabrafenib)

Therefore, using Jaccard similarity on the syllables of the drug names themselves may place drugs that are closely related to each other with a single metric.

FIG. 7C shows an example of a portion of a table used to create a drug similarity matrix. Table 730 contains two columns, treatment 731 and treatment2 732, which each enumerate all of the drugs or treatments, including all variants (brand names, generics, misspellings, etc.). The last column net sim 737 may be the ensemble score. All remaining columns 733, 734, 735, and 736 may be the various components of the similarity metric.

As an example, row 750 may compare two drugs, cyclophosphamide and fludarabine. Because these two drugs are often used in combination in clinical trials, they have a non-zero Jaccard similarity of 0.273. However, the cosine string distance is zero because the names of the two drugs are highly dissimilar.

In general, the ensemble score can be an arbitrary function of the components. For example, it may be a weighted sum, it may depend conditionally upon some of the component values, etc.

Returning again to FIG. 3, the Latent Semantic Analysis (LSA) Module 330 may also create mappers, but potentially more complex ones. This module can use tools such as LDA to not only map from ngrams to topics, but also from topics to subtopics, and to employ “chaining” to, for example, map from topics to drugs, or diseases to drugs, allowing second or higher order interactions between topics and subtopics. Chaining may be performed using multiplication of the matrix 321 from the Topic Model Module 320 by the matrix 331 of the LSA Module 330.

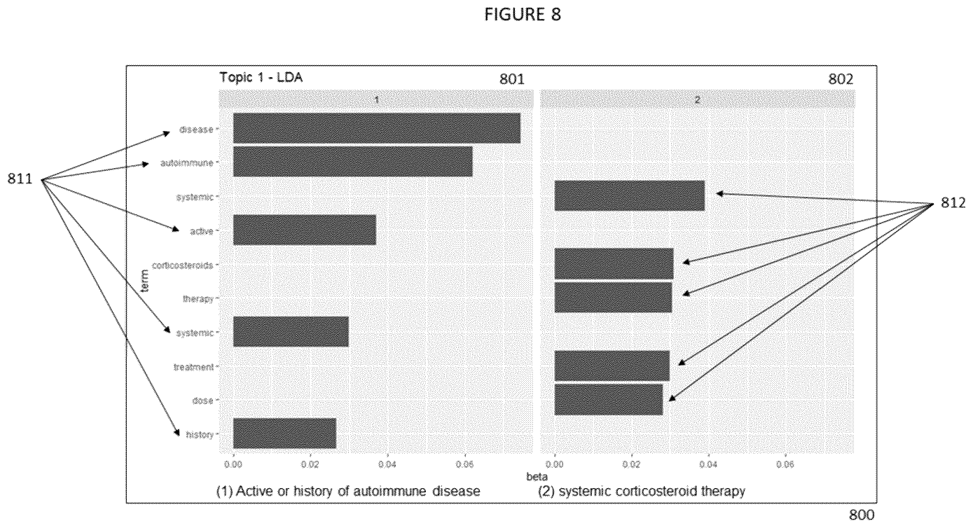

FIG. 8 provides an example of using the LSA module to create subtopics, using the same language terms that were used in FIG. 6. Window 800 may be divided into two panes, and Latent Dirichlet Allocation may be used, with the hyperparameters configured to divide the corpus into two parts. The keywords may be shown in order of frequency. In pane 801, one set of words 811 are allocated; in pane 802, another set of words 812 are allocated.

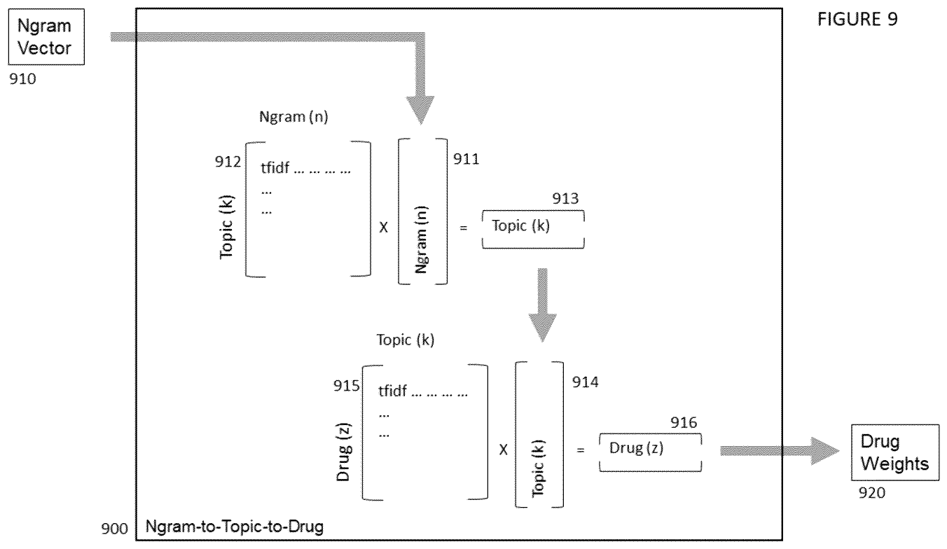

FIG. 9 shows an example of an ngram_to_drug mapper 900 of type “Ngram-to-Topic-to-Drug,” generated by the LSA module. It may take as input a weighted vector of all ngrams 910 (for example, the case summary for a particular patient, such as the patient case summary 211 of FIG. 2). It may then coerce this input into column format 911 for multiplication with the Topic-Ngram TF-IDF matrix 912 that was produced by the Topic Model Module 320 of FIG. 3. The result may be a vector of topic weights 913 as to how likely each topic applies to this particular document (e.g., in this case, the patient case summary).

Next, topic vector 913 may be transposed to columnar form 914, so that it can be multiplied by Drug-Topic TF-IDF matrix 915 to produce vector 916 of weighted drug rankings. Matrix 915 may be produced by the Topic Model Module 320 of FIG. 3 using data created as part of the Topic modeling and refinement process 315. Vector 916 may be outputted as the Drug Weights 920 of the Ngram-to-Topic-to-Drug mapper output.

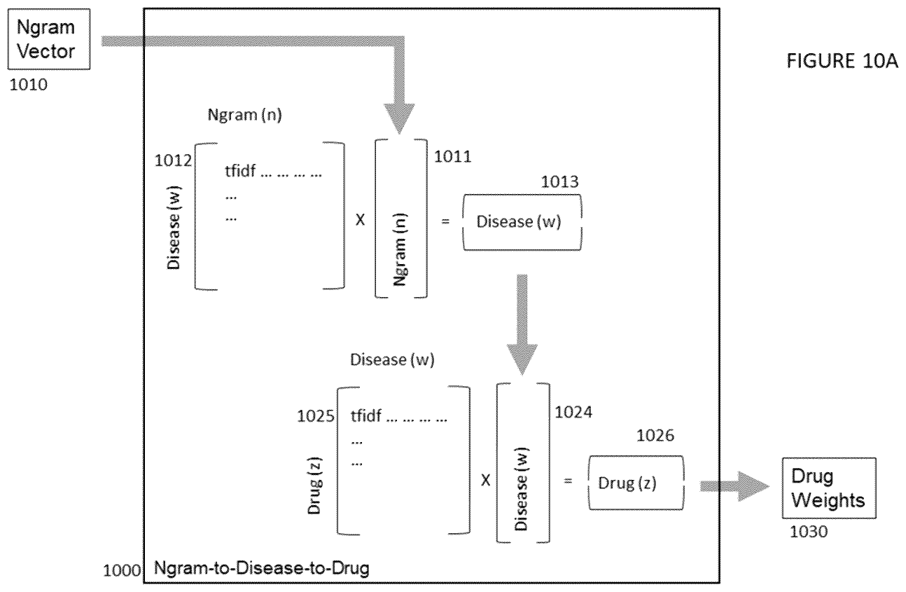

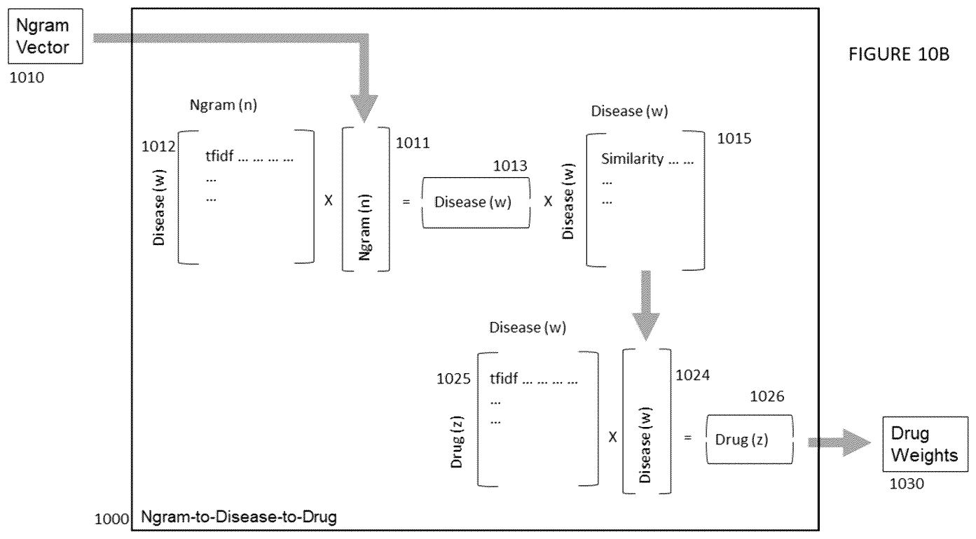

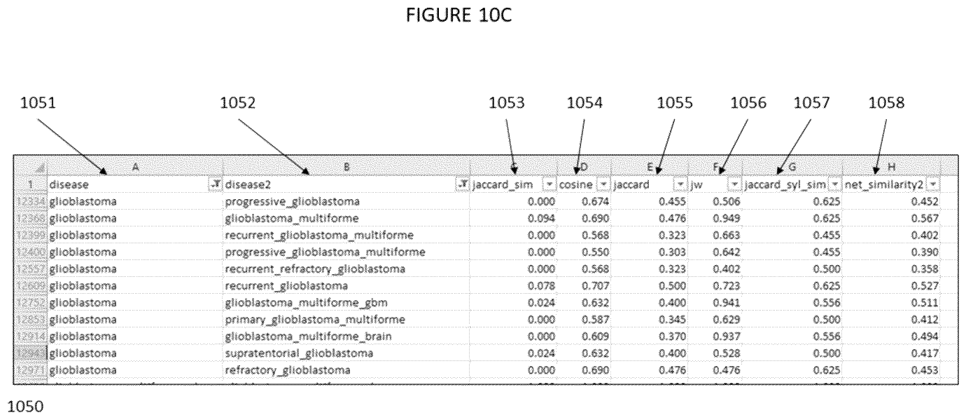

Similarly, FIG. 10A shows an example of an ngram_to_drug mapper 1000 of type “Ngram-to-Disease-to-Drug,” generated by the LSA module. It may take as input a weighted vector of all ngrams 1010 (for example, the case summary for a particular patient, such as the patient case summary 211 of FIG. 2). It may then coerce this input into column format 1011 for multiplication with the Disease-Ngram TF-IDF matrix 1012, which may be produced by the Topic Model Module 320 of FIG. 3 using data created as part of the Topic modeling and refinement process 315. The result may be a vector of disease weights 1013 as to how likely each disease applies to this particular document (e.g., in this case, the patient case summary), and thus, how likely this patient is to have this disease.

Next, topic vector 1013 may be transposed to columnar form 1024, so that it can be multiplied by Drug-Disease TF-IDF matrix 1025 to produce vector 1026 of weighted drug rankings. Matrix 1025 may be produced by the Topic Model Module 320 of FIG. 3 using data created as part of the Topic modeling and refinement process 315. Vector 1026 may be outputted as the Drug Weights 1030 of the Ngram-to-Disease-to-Drug mapper output.

As was demonstrated previously, such a mapper may not perform optimally, owing to the fact that doctors sometimes misdiagnose diseases, there are categories of diseases that are widely overlapping and hard to differentially diagnose, such as glioblastoma multiforme and supratentorial glioma, there are abbreviations (GBM=glioblastoma multiforme), progress from one disease to another related disease such as anaplastic astrocytoma into glioblastoma multiforme, source documents for training contain misspellings, and so forth.

Thus, FIG. 10B illustrates an embodiment of the “Ngram-to-Disease-to-Drug” mapper. It may be identical in function to that of FIG. 10A from the input Ngram Vector 1010 up until the point of the drug weightings row vector 1013. However, starting at this point, vector 1013 may be multiplied by a square matrix of the same dimension as vector 1013's length, the disease similarity matrix 1015, to adjust the weights for the diseases that are to be transposed to columnar form 1024. These may then be multiplied, as before, by the Drug-Disease TF-IDF matrix 1025 to produce vector 1026 of weighted drug rankings, which may be outputted as the Drug Weights 1030 from the mapper.

The disease similarity matrix 1015 may be computed in a manner similar to that for drug similarity, including (by way of example, but not limited to) one or more of the following:
A metric of overlap between occurrence of the two diseases in a clinical trial, summed over the space of trials;
Cosine similarity between terms defining the disease, where the cosine between two terms is the angle between the vector representation of the components of the terms;
Jaccard similarity between terms defining the disease;
Jaro-Winkler distance between the terms (possible with other measures for an ensemble score); and
Jaccard syllable similarity between disease names.

Again, an ensemble score may be computed using an arbitrary function of these metrics.

FIG. 10C shows an example of a portion of a table used to create a disease similarity matrix. Table 1050 may contain two columns, disease 1051 and disease2 1052, which may each enumerate all of the drugs/treatments, including all variants (brand names, generics, misspellings, etc.). The last column net similarity2 1058 may be the ensemble score. All remaining columns 1053, 1054, 1055, 1056, and 1057 may be the various components of the similarity metric.

In some embodiments, these types of chaining mappers can make use of much richer relationships among the various entity types in the ontology space: patients, diseases, features, genomic or other biomarkers, drugs, etc. The chaining need not stop at two levels: Ngram-to-Biomarker-to-Disease-to-Drug, or ngram-to-rationale-to-topic-to-drug are two examples of 3-chains.

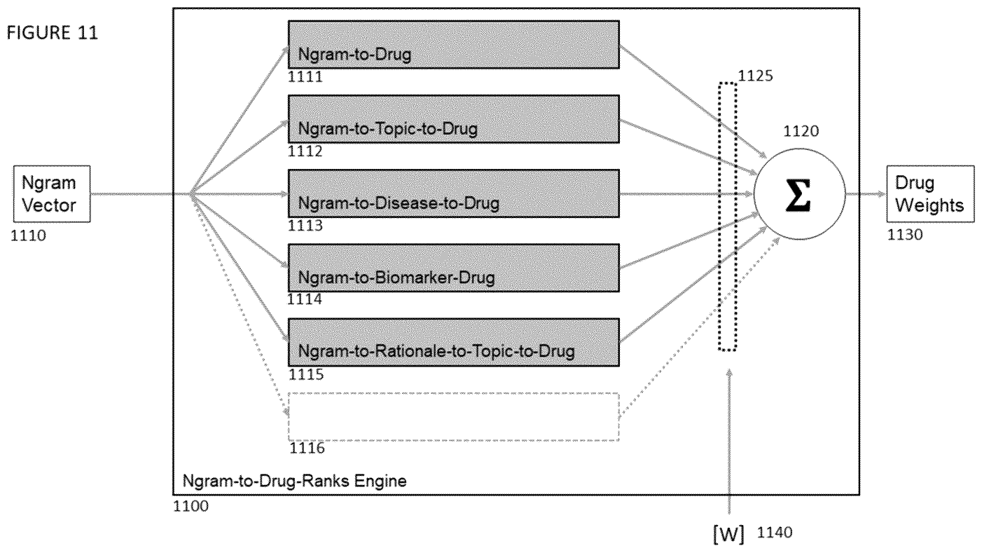

FIG. 11 illustrates an example of how the outputs of the mappers are combined to produce a final ranking of the suggested drug treatments, given the input document. The Ngram-to-Drug-Ranks Engine 1100 may take as input the weighted vector of all ngrams 1110, and may distribute it to all the mappers registered with the Engine. This example shows 5 mappers registered 1111, 1112, 1113, 1115 and 1115. In addition, the dashed box 1116 may indicate that the architecture is dynamic and extensible, and that additional mappers can be registered and added at any time.

Since the rankings of the suggested drugs may be relative, the final rankings that are outputted 1130 may be determined simply by summing the contributions of each of the mappers, via summing node 1120. Because the output of this process may be used by other algorithms that may expect consistency of scaling (e.g., the absolute value of the vector weights should not increase if more mappers are added), some embodiments include a normalization or scaling operation in the summation node 1120, e.g., such that sum of the weights in the drug weights vector 1130 ranges from 0 to 1 based on the content of the structured and unstructured case representation.

Additionally, the various mappers may not contribute equally to the summation process. Therefore, in some embodiments, a weighting vector 1125 may be included, which may multiply each incoming value to the summation node 1120 by a constant value, allowing the relative contributions of the mappers to be set. This can be controlled by an external weights vector [W] 1140. If this input is absent, it may be assumed to be a vector of all 1's.

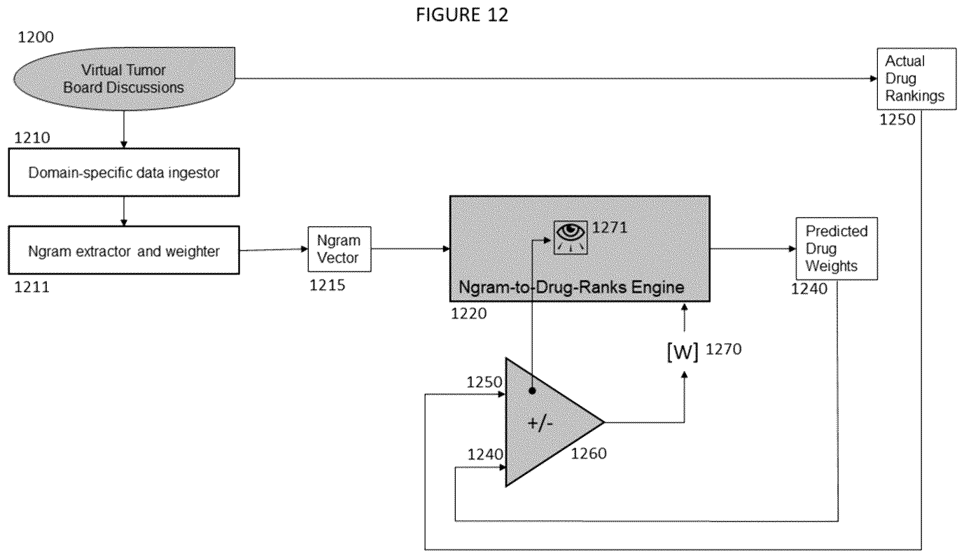

FIG. 12 shows an example of how the external weights vector can be used within a machine learning loop to optimize the values within [W]. This example assumes only one source of data (recommendations from Virtual Tumor Board Discussions 1200) is used for a supervised learning loop. A goal may be to adjust the weighting values so that the predicted drug weights lead to rankings that are as close to the actual drug rankings as possible.

For some set of tumor board discussions, the patient data may be fed through the appropriate data ingestor 1210, plus ngram extractor and weighter 1211 to create the ngram vector 1215. This may be fed into the Ngram-to-Drug-Ranks Engine 1220 which is tuned with whatever the current weights [W] 1270 are, producing a set of predicted weights 1240 for a broad range of drugs or treatments.

The actual tumor board may consider only a small set of drugs or treatments 1250 (e.g., four to eight), and rank orders those. Both the ranked treatments 1250 and the predicted ranks 1240 may be fed into a comparator 1260. The comparator may removes elements from vector 1240 which are not present in vector 1250, allowing it to compare the two vectors. It can then use various machine learning methods to adjust the weights [W] 1270 to optimize the system. Since the entire system may be open, there may be no need to treat the Ngram-to-Drug-Ranks Engine 1220 as a black box. The comparator can be much more efficient in learning the optimal weights if it has visibility 1271 into the inner workings of the Engine.

The choice of machine learning method for the comparator 1260 may depend on the number of training examples. Since the feature space may be quite large, a small number of training examples may not be amenable to some methods. For large numbers of training examples, techniques like XGBoost can be appropriate; for smaller numbers of training examples, methods like Bayesian Rejection Sampling may be more apropos.

Once a Bayesian updating process has been established for learning the hyperparameters of the language model from expert feedback, the system can be further refined through applications of active learning techniques, including, but not limited to, Thompson Sampling, upper confidence bound sampling, or knowledge gradient sampling. Such techniques define policies for choosing actions to achieve some specified reward. In context, the reward can be quantified with a metric between model-predicted treatment ranking and the observed treatment ranking. The Kendall tau distance is one such metric, though other metrics, such as those defined by any measure of rank correlation, may also be applicable.

With a specified reward metric, the system can define a space of actions which, when taken, results in different combinations of case features and treatment features. For example, the system can make the decision of what (if any) additional treatment options to include in the set of possible treatment options for experts to review. This decision may add additional information to be gained from experts per each ranking, but may increase the burden on experts. Active learning policies can help optimize this trade-off by selecting actions that maximize a metric of information-theoretic value.

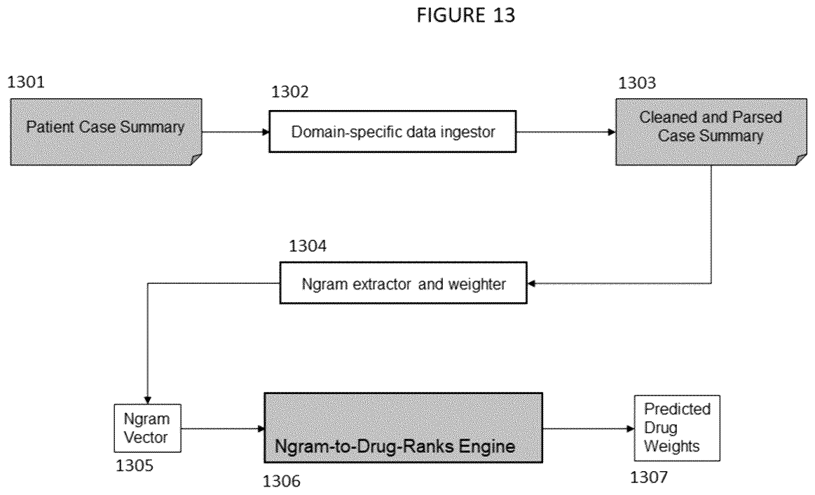

Whether the weights vector is used as all 1's or is optimized, an example of the runtime configuration is as shown in FIG. 13. A document such as a Patient Case Summary 1301 may be parsed and cleaned using a domain-specific data ingestor 1302, resulting in a cleaned and parsed case summary 1303. This may then be fed to the ngram extractor and weighter 1304, which may produce a vector 1305 of all the ngrams the system knows about, weighted according to relevance to this document (case summary). This vector may serve as input to the Ngram-to-Drug-Ranks Engine 1306, which may produce a vector of predicted drug weights 1307. Again, the label “drug,” may refer to any patient treatment, including, but not limited to drugs, surgery, radiation, diets, combination therapy, etc.

The Patient Case Summary 1301 of some embodiments may contain both structured and unstructured data. The structured elements may come from defined fields of an Electronic Health Record (EHR) or Electronic Data Capture (EDC) system, and may contain information such as diagnosis, stage and grade of disease, medications, vitals, laboratory results, etc. The unstructured elements may be attached as documents within an EHR or EDC system, but in order to extract the information with these documents, they may need to be parsed and processed. Within these elements, information such as pathology and histology of the disease, assessment of disease progression according to imaging studies, and other such findings subject to human expertise and assessment may be located.

When the drug weights vector is sorted from largest weight to smallest, the top values may provide a ranked list of treatment options that best match the patient's needs, based upon the particulars of the patient's case summary.

In addition to using the system of the present disclosure to produce a set of specific treatment options for a specific patient given the patient summary, it is also possible to employ the system to create “generic” options libraries for classes of patients who fit certain profiles. For example, one may wish to create an options library for pancreatic cancer patients with disease that is metastatic to the liver, or for midline glioma patients.

In order to produce such a library, the operations may comprise:
1. Collect a large enough representative sample of patient case summaries from a cohort of patients who have the disease of interest, comorbidities of interest, etc.;
2. Generate ranked treatment options for each such patient;
3. Create a list of each treatment and the count of how many times it appeared in the ranked treatment options that were generated; and,
4. Sort the newly created list (e.g., from most references to fewest).

### Computer Systems

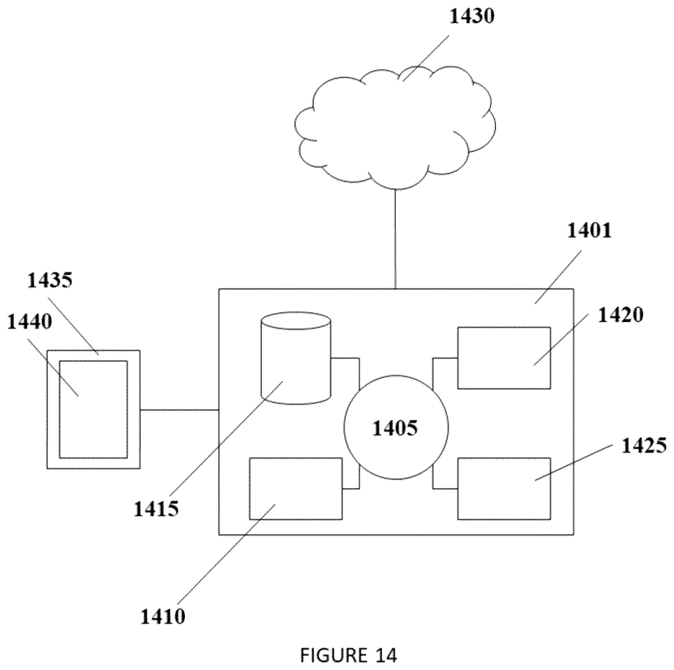

The present disclosure provides computer systems that are programmed to implement methods of the disclosure. FIG. 14 shows a computer system 1401 that is programmed or otherwise configured to implement systems and methods of the present disclosure. The computer system 1401 can implement and regulate various aspects of the systems and methods of the present disclosure. The computer system 1401 can be an electronic device of a user or a computer system that is remotely located with respect to the electronic device. The electronic device can be a mobile electronic device. For example, the computer system can be an electronic device of a sender or recipient, or a computer system that is remotely located with respect to the sender or recipient.

The computer system 1401 includes a central processing unit (CPU, also “processor” and “computer processor” herein) 1405, which can be a single core or multi core processor, or a plurality of processors for parallel processing. The computer system 1401 also includes memory or memory location 1410 (e.g., random-access memory, read-only memory, flash memory), electronic storage unit 1415 (e.g., hard disk), communication interface 1420 (e.g., network adapter) for communicating with one or more other systems, and peripheral devices 1425, such as cache, other memory, data storage and/or electronic display adapters. The memory 1410, storage unit 1415, interface 1420 and peripheral devices 1425 are in communication with the CPU 1405 through a communication bus (solid lines), such as a motherboard. The storage unit 1415 can be a data storage unit (or data repository) for storing data. The computer system 1401 can be operatively coupled to a computer network (“network”) 1430 with the aid of the communication interface 1420. The network 1430 can be the Internet, an internet and/or extranet, or an intranet and/or extranet that is in communication with the Internet. The network 1430 in some cases is a telecommunication and/or data network. The network 1430 can include one or more computer servers, which can enable distributed computing, such as cloud computing. The network 1430, in some cases with the aid of the computer system 1401, can implement a peer-to-peer network, which may enable devices coupled to the computer system 1401 to behave as a client or a server.

The CPU 1405 can execute a sequence of machine-readable instructions, which can be embodied in a program or software. The instructions may be stored in a memory location, such as the memory 1410. The instructions can be directed to the CPU 1405, which can subsequently program or otherwise configure the CPU 1405 to implement methods of the present disclosure. Examples of operations performed by the CPU 1405 can include fetch, decode, execute, and writeback.

The CPU 1405 can be part of a circuit, such as an integrated circuit. One or more other components of the system 1401 can be included in the circuit. In some cases, the circuit is an application specific integrated circuit (ASIC).

The storage unit 1415 can store files, such as drivers, libraries and saved programs. The storage unit 1415 can store user data, e.g., user preferences and user programs. The computer system 1401 in some cases can include one or more additional data storage units that are external to the computer system 1401, such as located on a remote server that is in communication with the computer system 1401 through an intranet or the Internet.

The computer system 1401 can communicate with one or more remote computer systems through the network 1430. For instance, the computer system 1401 can communicate with a remote computer system of a user (e.g., sender, recipient, etc.). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1401 via the network 1430.

Methods as described herein can be implemented by way of machine (e.g., computer processor) executable code stored on an electronic storage location of the computer system 1401, such as, for example, on the memory 1410 or electronic storage unit 1415. The machine executable or machine readable code can be provided in the form of software. During use, the code can be executed by the processor 1405. In some cases, the code can be retrieved from the storage unit 1415 and stored on the memory 1410 for ready access by the processor 1405. In some situations, the electronic storage unit 1415 can be precluded, and machine-executable instructions are stored on memory 1410.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 1401, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1401 can include or be in communication with an electronic display 1435 that comprises a user interface (UI) 1440 for providing, for example, an instructions panel of document restructuring, input/output preview, etc. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1405.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-100. (canceled)

101. A computer-implemented method for generating an individual recommendation for medical treatment of a subject, the method comprising:
(a) receiving, from a first set of distinct sources, first information relating to a set of diseases or disorders encompassing a medical domain;
(b) processing the first information relating to the set of diseases or disorders to generate a first document corpus, wherein processing the first information comprises parsing structured information or textual information of the first information;
(c) receiving, from a second set of distinct sources, second information relating to a disease or disorder of the subject, wherein the second information comprises a clinical information of the subject;
(d) processing the second information relating to the disease or disorder of the subject to generate a second document corpus, wherein processing the second information comprises parsing structured information or textual information of the second information; and
(e) generating a ranked set of candidate treatments for treating the disease or disorder of the subject, based at least in part on processing the first document corpus with the second document corpus.

102. The method of claim 101, wherein (a) further comprises receiving, from a remote server, the first information relating to the set of diseases or disorders encompassing the medical domain; or wherein (c) further comprises receiving, from a remote server, the second information relating to the disease or disorder of the subject.

103. The method of claim 101, wherein the disease or disorder is cancer.

104. The method of claim 101, wherein the first information relating to the set of diseases or disorders comprises clinical trial information, a tumor board discussion, a case summary or report, and/or outcomes reported by subjects.

105. The method of claim 101, wherein the second information relating to the disease or disorder of the subject comprises diagnosis, stage and grade of disease, medications, vitals, laboratory results, clinical trial information, tumor board discussions, a case summary or report, and/or an outcome reported by the subject.

106. The method of claim 101, wherein the clinical information of the subject comprises a case summary of the disease or disorder of the subject.

107. The method of claim 101, wherein (b) further comprises parsing the structured information or textual information of the first information according to an ontology of treatment concepts, or wherein (d) further comprises parsing the structured information or textual information of the second information according to an ontology of treatment concepts.

108. The method of claim 101, wherein (b) further comprises parsing the structured information or textual information of the first information to discover concepts pertaining to at least one topic selected from clinical trial information, a tumor board discussion, a case summary or report, and outcomes reported subjects; or wherein (d) further comprises parsing the structured information or textual information of the second information to discover concepts pertaining to at least one topic selected from diagnosis, stage and grade of disease, medications, vitals, laboratory results, clinical trial information, a tumor board discussion, a case summary or report, and an outcome reported by the subject.

109. The method of claim 101, wherein (b) further comprises generating a topic space for documents received from the first set of distinct sources, or wherein (d) further comprises generating a topic space for documents received from the second set of distinct sources.

110. The method of claim 101, wherein (b) further comprises associating a topic with a specific document received from a distinct source of the first set of distinct sources, or wherein (d) further comprises associating a topic with a specific document received from a distinct source of the second set of distinct sources.

111. The method of claim 101, wherein (b) further comprises parsing the structured information or textual information of the first information using one or more algorithms selected from the group consisting of a structured data parser, a text recognition algorithm, a regular expressions algorithm, a pattern recognition algorithm, an imaging recognition algorithm, a natural language processing algorithm, an optical character recognition algorithm, a term frequency-inverse document frequency (TF-IDF) algorithm, and a bag-of-words algorithm; or wherein (d) further comprises parsing the structured information or textual information of the second information using one or more algorithms selected from the group consisting of a structured data parser, a text recognition algorithm, a regular expressions algorithm, a pattern recognition algorithm, an imaging recognition algorithm, a natural language processing algorithm, an optical character recognition algorithm, a term frequency-inverse document frequency (TF-IDF) algorithm, and a bag-of-words algorithm.

112. The method of claim 101, wherein (b) further comprises determining, based at least in part on the parsing in (b), whether the structured information or textual information of the first information corresponds to a clinical trials database, a clinical trial arm description, a genomics database, a clinical care guideline document, a case series document, a drug database, an imaging report, a pathology report, a clinic note, a progress note, a genomics report, a laboratory test report, a diagnostic report, or a prognostic report; or wherein (d) further comprises determining, based at least in part on the parsing in (d), whether the structured information or textual information of the second information corresponds to an imaging report, a pathology report, a clinic note, a progress note, a genomics report, a laboratory test report, a diagnostic report, or a prognostic report.

113. The method of claim 101, wherein parsing the structured information or textual information of the first or second information comprises at least one of case converting the structured information or textual information of the first or second information, removing special characters or stop words from the structured information or textual information of the first or second information, tokenizing the structured information or textual information of the first or second information, and parsing the structured information or textual information of the first or second information using a parser.

114. The method of claim 101, wherein parsing the structured information or textual information of the first or second information comprises filtering the structured information or textual information of the first or second information for at least one disease state, a treatment for the at least one disease state, or clinical trials associated with the at least one disease state or the treatment for the at least one disease state.

115. The method of claim 101, wherein parsing the structured information or textual information of the first or second information comprises extracting and standardizing inclusion or exclusion criteria.

116. The method of claim 101, wherein parsing the structured information or textual information of the first or second information comprises labeling the structured information or textual information of the first or second information with labels.

117. The method of claim 101, wherein parsing the structured information or textual information of the first or second information comprises performing named entity recognition.

118. The method of claim 101, wherein (b) further comprises generating a set of sub-corpuses from the first document corpus, or wherein (d) further comprises generating a set of sub-corpuses from the second document corpus.

119. The method of claim 101, wherein (b) further comprises performing topic modeling.

120. The method of claim 119, wherein the topic modeling in (b) comprises use of at least one of Biterm Topic Modeling (BTM), Latent Dirichlet Allocation (LDA), and Term Frequency-Inverse Document Frequency (TF-IDF) analysis.

121. The method of claim 120, wherein the topic modeling in (b) comprises generating ngrams of frequently occurring word combinations in the first information.

122. The method of claim 121, wherein (e) further comprises mapping the ngrams of at least one of the first information and the second information to a set of candidate treatments, and generating the ranked set of candidate treatments based at least in part on the mapping.

123. The method of claim 122, wherein the mapping comprises partitioning at least one of the first document corpus and the second document corpus based on a topic.

124. The method of claim 122, wherein the mapping comprises performing a plurality of mappings comprising at least a first mapping from the ngrams to a topic, subtopic, or disease, and a second mapping from the topic, the subtopic, or the disease to the set of candidate treatments.

125. The method of claim 119, wherein the topic modeling in (b) comprises partitioning the first document corpus into a set of topics or subtopics.

126. The method of claim 119, wherein the topic modeling in (b) comprises associating relationships between ngrams and treatments, ngrams and disease state, ngrams and treatment rationales, or a combination thereof.

127. The method of claim 101, wherein processing the first document corpus with the second document corpus in (e) further comprises comparing the first document corpus and second document corpus to each other.

128. The method of claim 101, further comprising performing at least one iteration of (a) and (b) to incorporate new or updated medical information into the first document corpus.

129. A system for generating an individual recommendation for medical treatment of a subject, comprising:
a database that is configured to (i) receive from a first set of distinct sources, first information relating to a set of diseases or disorders encompassing a medical domain, and (ii) receive from a second set of distinct sources, second information relating to a disease or disorder of the subject, wherein the second information comprises a clinical information of the subject; and one or more computer processors operatively coupled to the database, wherein the one or more computer processors are individually or collectively programmed to:
(a) process the first information relating to the set of diseases or disorders to generate a first document corpus, wherein processing the first information comprises parsing structured information or textual information of the first information;
(b) process the second information relating to the disease or disorder of the subject to generate a second document corpus, wherein processing the second information comprises parsing structured information or textual information of the second information; and
(c) generate a ranked set of candidate treatments for treating the disease or disorder of the subject, based at least in part on processing the first document corpus with the second document corpus.

130. A non-transitory computer-readable medium comprising machine-executable code that, upon execution by one or more computer processors, implements a method for generating an individual recommendation for medical treatment of a subject, the method comprising:
(a) receiving, from a first set of distinct sources, first information relating to a set of diseases or disorders encompassing a medical domain;
(b) processing the first information relating to the set of diseases or disorders to generate a first document corpus, wherein processing the first information comprises parsing structured information or textual information of the first information;
(c) receiving, from a second set of distinct sources, second information relating to a disease or disorder of the subject, wherein the second information comprises a clinical information of the subject;
(d) processing the second information relating to the disease or disorder of the subject to generate a second document corpus, wherein processing the second information comprises parsing structured information or textual information of the second information; and
(e) generating a ranked set of candidate treatments for treating the disease or disorder of the subject, based at least in part on processing the first document corpus with the second document corpus.
